SEQUENCE LISTING (154 sequences)

SEQ ID NO: 1 - RNA, 19 nt, synthetic construct
  source 1..19: mol_type = other RNA
tgtactctctgaggtgctc

SEQ ID NO: 2 - RNA, 19 nt, synthetic construct
  source 1..19: mol_type = other RNA
acgcagataagaaccagtt

SEQ ID NO: 3 - RNA, 19 nt, synthetic construct
  source 1..19: mol_type = other RNA
catcaagtcagccatcagc

SEQ ID NO: 4 - RNA, 19 nt, synthetic construct
  source 1..19: mol_type = other RNA
gagtcaccctcctggaaac

SEQ ID NO: 5 - RNA, 20 nt, synthetic construct
  source 1..20: mol_type = other RNA
actccactgcactccagtct

SEQ ID NO: 6 - RNA, 20 nt, synthetic construct
  source 1..20: mol_type = other RNA
tctgtgggggacctgcactg

SEQ ID NO: 7 - RNA, 20 nt, synthetic construct
  source 1..20: mol_type = other RNA
ggggcgccagttgtgtctcc

SEQ ID NO: 8 - RNA, 20 nt, synthetic construct
  source 1..20: mol_type = other RNA
acaccattgccaccaccatt

SEQ ID NO: 9 - RNA, 19 nt, synthetic construct
  source 1..19: mol_type = other RNA
gctagggatgaagaataaa

SEQ ID NO: 10 - RNA, 19 nt, synthetic construct
  source 1..19: mol_type = other RNA
ttgaccaatagccttgaca

SEQ ID NO: 11 - RNA, 19 nt, synthetic construct
  source 1..19: mol_type = other RNA
tgcaaatatctgtctgaaa

SEQ ID NO: 12 - RNA, 19 nt, synthetic construct
  source 1..19: mol_type = other RNA
aaattagcagtatcctctt

SEQ ID NO: 13 - RNA, 20 nt, synthetic construct
  source 1..20: mol_type = other RNA
acttcaggttcaaagaagcc

SEQ ID NO: 14 - RNA, 20 nt, synthetic construct
  source 1..20: mol_type = other RNA
ccctgggtggggaaaaccag

SEQ ID NO: 15 - RNA, 20 nt, synthetic construct
  source 1..20: mol_type = other RNA
ttttccccacccagggccta

SEQ ID NO: 16 - RNA, 20 nt, synthetic construct
  source 1..20: mol_type = other RNA
agggagaacggggcctaccg

SEQ ID NO: 17 - RNA, 20 nt, synthetic construct
  source 1..20: mol_type = other RNA
cagacatctaataccacggt

SEQ ID NO: 18 - RNA, 20 nt, synthetic construct
  source 1..20: mol_type = other RNA
agtgataagacacccgcttt

SEQ ID NO: 19 - RNA, 20 nt, synthetic construct
  source 1..20: mol_type = other RNA
gcatgacaaaggtgccgtga

SEQ ID NO: 20 - RNA, 20 nt, synthetic construct
  source 1..20: mol_type = other RNA
gtgccgtgatggttctgtcc

SEQ ID NO: 21 - RNA, 20 nt, synthetic construct
  source 1..20: mol_type = other RNA
ggaggaacatgcttcggaac

SEQ ID NO: 22 - RNA, 20 nt, synthetic construct
  source 1..20: mol_type = other RNA
cctgccttttgggcagttaa

SEQ ID NO: 23 - RNA, 20 nt, synthetic construct
  source 1..20: mol_type = other RNA
tcggcctttaactgcccaaa

SEQ ID NO: 24 - RNA, 20 nt, synthetic construct
  source 1..20: mol_type = other RNA
ggtctgccggaaggtctaca

SEQ ID NO: 25 - RNA, 20 nt, synthetic construct
  source 1..20: mol_type = other RNA
aatatgtcacattctgtctc

SEQ ID NO: 26 - RNA, 20 nt, synthetic construct
  source 1..20: mol_type = other RNA
ggactatgggaggtcactaa

SEQ ID NO: 27 - RNA, 20 nt, synthetic construct
  source 1..20: mol_type = other RNA
gaaggttacacagaaccaga

SEQ ID NO: 28 - RNA, 20 nt, synthetic construct
  source 1..20: mol_type = other RNA
gccctgtaagcatcctgctg

SEQ ID NO: 29 - RNA, 20 nt, synthetic construct
  source 1..20: mol_type = other RNA
ccgttctatgagatgctccg

SEQ ID NO: 30 - RNA, 20 nt, synthetic construct
  source 1..20: mol_type = other RNA
gcaagtaggctcctcggcag

SEQ ID NO: 31 - RNA, 20 nt, synthetic construct
  source 1..20: mol_type = other RNA
cacgcacggggactgcaagc

SEQ ID NO: 32 - RNA, 20 nt, synthetic construct
  source 1..20: mol_type = other RNA
cctctttgcaagtgctgcac

SEQ ID NO: 33 - RNA, 20 nt, synthetic construct
  source 1..20: mol_type = other RNA
attcaagggaagcgagcgct

SEQ ID NO: 34 - RNA, 20 nt, synthetic construct
  source 1..20: mol_type = other RNA
cacaggcgaggttgtcaacg

SEQ ID NO: 35 - RNA, 20 nt, synthetic construct
  source 1..20: mol_type = other RNA
ttattactacttgccctcag

SEQ ID NO: 36 - RNA, 20 nt, synthetic construct
  source 1..20: mol_type = other RNA
tcaagctagtcttcagcaaa

SEQ ID NO: 37 - RNA, 20 nt, synthetic construct
  source 1..20: mol_type = other RNA
ttaaactttaattgcattaa

SEQ ID NO: 38 - RNA, 20 nt, synthetic construct
  source 1..20: mol_type = other RNA
ggagtgctgttagacaacaa

SEQ ID NO: 39 - RNA, 20 nt, synthetic construct
  source 1..20: mol_type = other RNA
taaatcgttactcagtcatc

SEQ ID NO: 40 - RNA, 20 nt, synthetic construct
  source 1..20: mol_type = other RNA
ccacctatcccattattcat

SEQ ID NO: 41 - RNA, 20 nt, synthetic construct
  source 1..20: mol_type = other RNA
accactctcaagttgtccat

SEQ ID NO: 42 - RNA, 20 nt, synthetic construct
  source 1..20: mol_type = other RNA
ggaaaggtgagtatattaca

SEQ ID NO: 43 - RNA, 20 nt, synthetic construct
  source 1..20: mol_type = other RNA
tatatttaggggcacgaact

SEQ ID NO: 44 - RNA, 20 nt, synthetic construct
  source 1..20: mol_type = other RNA
acacggacttcatgacacat

SEQ ID NO: 45 - RNA, 20 nt, synthetic construct
  source 1..20: mol_type = other RNA
tgtcagaaaccattagcggt

SEQ ID NO: 46 - RNA, 20 nt, synthetic construct
  source 1..20: mol_type = other RNA
agcctcattaggatagcagt

SEQ ID NO: 47 - RNA, 20 nt, synthetic construct
  source 1..20: mol_type = other RNA
ctgctgacattttacaaatg

SEQ ID NO: 48 - RNA, 20 nt, synthetic construct
  source 1..20: mol_type = other RNA
ttacagtgtaaaaacgaatt

SEQ ID NO: 49 - RNA, 20 nt, synthetic construct
  source 1..20: mol_type = other RNA
agttgccgtttatgtactga

SEQ ID NO: 50 - RNA, 20 nt, synthetic construct
  source 1..20: mol_type = other RNA
aggcaaaaccaagactatgc

SEQ ID NO: 51 - RNA, 20 nt, synthetic construct
  source 1..20: mol_type = other RNA
gcttctgcatgcaaggttgt

SEQ ID NO: 52 - RNA, 20 nt, synthetic construct
  source 1..20: mol_type = other RNA
aattttagctttacaaaggg

SEQ ID NO: 53 - RNA, 20 nt, synthetic construct
  source 1..20: mol_type = other RNA
ttgacctttaatggtaagct

SEQ ID NO: 54 - RNA, 20 nt, synthetic construct
  source 1..20: mol_type = other RNA
gcttgagagtgtctgttagc

SEQ ID NO: 55 - RNA, 20 nt, synthetic construct
  source 1..20: mol_type = other RNA
tgaatgttctttactcaaat

SEQ ID NO: 56 - RNA, 20 nt, synthetic construct
  source 1..20: mol_type = other RNA
gatgagcccaggctaattca

SEQ ID NO: 57 - RNA, 20 nt, synthetic construct
  source 1..20: mol_type = other RNA
gttttatcagcacaactcct

SEQ ID NO: 58 - RNA, 20 nt, synthetic construct
  source 1..20: mol_type = other RNA
acaacaattacagggggagg

SEQ ID NO: 59 - RNA, 20 nt, synthetic construct
  source 1..20: mol_type = other RNA
ggccaccccatctataaaca

SEQ ID NO: 60 - RNA, 20 nt, synthetic construct
  source 1..20: mol_type = other RNA
gaggttacctatgtactttt

SEQ ID NO: 61 - RNA, 20 nt, synthetic construct
  source 1..20: mol_type = other RNA
gaaatgtcactaattaggag

SEQ ID NO: 62 - RNA, 20 nt, synthetic construct
  source 1..20: mol_type = other RNA
ccagtgacctttgagtacac

SEQ ID NO: 63 - RNA, 20 nt, synthetic construct
  source 1..20: mol_type = other RNA
cggttggactggtctatcat

SEQ ID NO: 64 - RNA, 20 nt, synthetic construct
  source 1..20: mol_type = other RNA
gtgttgttatgaaacgaaac

SEQ ID NO: 65 - RNA, 20 nt, synthetic construct
  source 1..20: mol_type = other RNA
tatgaccactccctggtgca

SEQ ID NO: 66 - RNA, 20 nt, synthetic construct
  source 1..20: mol_type = other RNA
agtccttagctagtacgcac

SEQ ID NO: 67 - RNA, 20 nt, synthetic construct
  source 1..20: mol_type = other RNA
agggcgagcagagtttacac

SEQ ID NO: 68 - RNA, 20 nt, synthetic construct
  source 1..20: mol_type = other RNA
ggaacctgttactttaagcg

SEQ ID NO: 69 - RNA, 20 nt, synthetic construct
  source 1..20: mol_type = other RNA
aggcagagcgagcgagccag

SEQ ID NO: 70 - RNA, 20 nt, synthetic construct
  source 1..20: mol_type = other RNA
cgcaatcatcttctttaccc

SEQ ID NO: 71 - RNA, 20 nt, synthetic construct
  source 1..20: mol_type = other RNA
ctgtccattcaccataagaa

SEQ ID NO: 72 - RNA, 20 nt, synthetic construct
  source 1..20: mol_type = other RNA
gtgtaacttaatcccttatt

SEQ ID NO: 73 - RNA, 20 nt, synthetic construct
  source 1..20: mol_type = other RNA
atcaactattacttgtagat

SEQ ID NO: 74 - RNA, 20 nt, synthetic construct
  source 1..20: mol_type = other RNA
atggtcaagagccataattg

SEQ ID NO: 75 - RNA, 20 nt, synthetic construct
  source 1..20: mol_type = other RNA
aattaagtgacacaatttca

SEQ ID NO: 76 - RNA, 20 nt, synthetic construct
  source 1..20: mol_type = other RNA
gctgttttaaaagtacgggt

SEQ ID NO: 77 - RNA, 20 nt, synthetic construct
  source 1..20: mol_type = other RNA
aaggcggattcatgagtgga

SEQ ID NO: 78 - RNA, 20 nt, synthetic construct
  source 1..20: mol_type = other RNA
aagaagctggtacataggac

SEQ ID NO: 79 - RNA, 20 nt, synthetic construct
  source 1..20: mol_type = other RNA
actgcctgtaaaatctccca

SEQ ID NO: 80 - RNA, 20 nt, synthetic construct
  source 1..20: mol_type = other RNA
ccagccatagagaatacgca

SEQ ID NO: 81 - RNA, 20 nt, synthetic construct
  source 1..20: mol_type = other RNA
tctcagaagcatattggcag

SEQ ID NO: 82 - RNA, 20 nt, synthetic construct
  source 1..20: mol_type = other RNA
tatttgtttgtgcacgtagg

SEQ ID NO: 83 - RNA, 20 nt, synthetic construct
  source 1..20: mol_type = other RNA
gatacaaaccatttacctgg

SEQ ID NO: 84 - RNA, 20 nt, synthetic construct
  source 1..20: mol_type = other RNA
tcagctttgaaggttgcctt

SEQ ID NO: 85 - RNA, 20 nt, synthetic construct
  source 1..20: mol_type = other RNA
ttacatggacctgctttccc

SEQ ID NO: 86 - RNA, 20 nt, synthetic construct
  source 1..20: mol_type = other RNA
gagtcataagctccagtgca

SEQ ID NO: 87 - RNA, 20 nt, synthetic construct
  source 1..20: mol_type = other RNA
gtactcaatgttattaaagc

SEQ ID NO: 88 - RNA, 20 nt, synthetic construct
  source 1..20: mol_type = other RNA
gcacctaggtttcatggtcc

SEQ ID NO: 89 - RNA, 20 nt, synthetic construct
  source 1..20: mol_type = other RNA
ggagtcgtcccatactggag

SEQ ID NO: 90 - RNA, 20 nt, synthetic construct
  source 1..20: mol_type = other RNA
actacaaaagattacccttc

SEQ ID NO: 91 - RNA, 20 nt, synthetic construct
  source 1..20: mol_type = other RNA
gatggacccttctaaaatag

SEQ ID NO: 92 - RNA, 20 nt, synthetic construct
  source 1..20: mol_type = other RNA
acgtttgacatgggaaagca

SEQ ID NO: 93 - RNA, 20 nt, synthetic construct
  source 1..20: mol_type = other RNA
atcataggacttttgctact

SEQ ID NO: 94 - RNA, 20 nt, synthetic construct
  source 1..20: mol_type = other RNA
ctccttacatttagcagacc

SEQ ID NO: 95 - RNA, 20 nt, synthetic construct
  source 1..20: mol_type = other RNA
tctgcttcgcaacttttatt

SEQ ID NO: 96 - RNA, 20 nt, synthetic construct
  source 1..20: mol_type = other RNA
tgactttaagattcaactac

SEQ ID NO: 97 - RNA, 20 nt, synthetic construct
  source 1..20: mol_type = other RNA
aaatcaactgtttgcacatt

SEQ ID NO: 98 - RNA, 20 nt, synthetic construct
  source 1..20: mol_type = other RNA
aggtgactcccaagtaagaa

SEQ ID NO: 99 - RNA, 20 nt, synthetic construct
  source 1..20: mol_type = other RNA
catctgctgttaacacgatc

SEQ ID NO: 100 - RNA, 20 nt, synthetic construct
  source 1..20: mol_type = other RNA
gctggcttcttatgtgacag

SEQ ID NO: 101 - RNA, 20 nt, synthetic construct
  source 1..20: mol_type = other RNA
aggaagaactgtaaccgttc

SEQ ID NO: 102 - RNA, 20 nt, synthetic construct
  source 1..20: mol_type = other RNA
tgttgggtgtgatcacactg

SEQ ID NO: 103 - RNA, 20 nt, synthetic construct
  source 1..20: mol_type = other RNA
ccgtgtttcagtacatacta

SEQ ID NO: 104 - RNA, 20 nt, synthetic construct
  source 1..20: mol_type = other RNA
agctggttgtcatcaacaat

SEQ ID NO: 105 - RNA, 20 nt, synthetic construct
  source 1..20: mol_type = other RNA
ccaacaatgggactcaacat

SEQ ID NO: 106 - RNA, 20 nt, synthetic construct
  source 1..20: mol_type = other RNA
tcaaaagcgagcacctgtca

SEQ ID NO: 107 - RNA, 20 nt, synthetic construct
  source 1..20: mol_type = other RNA
accaaaacaatgggcctacc

SEQ ID NO: 108 - RNA, 20 nt, synthetic construct
  source 1..20: mol_type = other RNA
attagtttgctcatagagtg

SEQ ID NO: 109 - RNA, 20 nt, synthetic construct
  source 1..20: mol_type = other RNA
aaagttatatttatgctata

SEQ ID NO: 110 - RNA, 20 nt, synthetic construct
  source 1..20: mol_type = other RNA
gctgatgactacttatcgat

SEQ ID NO: 111 - DNA, 20 bp, synthetic construct
  source 1..20: mol_type = other DNA
caatgaccccttcattgacc

SEQ ID NO: 112 - DNA, 30 bp, synthetic construct
  source 1..30: mol_type = other DNA
cgaaagagaaagcgaaccagtatcgagaac

SEQ ID NO: 113 - DNA, 20 bp, synthetic construct
  source 1..20: mol_type = other DNA
ggaatccatggagggaagat

SEQ ID NO: 114 - DNA, 21 bp, synthetic construct
  source 1..21: mol_type = other DNA
gctgagtgaactgcactgtga

SEQ ID NO: 115 - DNA, 20 bp, synthetic construct
  source 1..20: mol_type = other DNA
gtctgggcaacaaagtgaga

SEQ ID NO: 116 - DNA, 19 bp, synthetic construct
  source 1..19: mol_type = other DNA
ggcatcaagtcagccatca

SEQ ID NO: 117 - DNA, 20 bp, synthetic construct
  source 1..20: mol_type = other DNA
cttcagcagttccacacact

SEQ ID NO: 118 - DNA, 21 bp, synthetic construct
  source 1..21: mol_type = other DNA
tgttagagcattctttgagga

SEQ ID NO: 119 - DNA, 21 bp, synthetic construct
  source 1..21: mol_type = other DNA
ttctccctccttttcctccac

SEQ ID NO: 120 - DNA, 20 bp, synthetic construct
  source 1..20: mol_type = other DNA
ttgattttggagggatctcg

SEQ ID NO: 121 - DNA, 27 bp, synthetic construct
  source 1..27: mol_type = other DNA
cgttgtgcatagtcgctgcttgatcgc

SEQ ID NO: 122 - DNA, 20 bp, synthetic construct
  source 1..20: mol_type = other DNA
tgttctcgctcaggtcagtg

SEQ ID NO: 123 - DNA, 20 bp, synthetic construct
  source 1..20: mol_type = other DNA
gaattctttgccgaaatgga

SEQ ID NO: 124 - DNA, 20 bp, synthetic construct
  source 1..20: mol_type = other DNA
caatggtgtctgtggaaggg

SEQ ID NO: 125 - DNA, 21 bp, synthetic construct
  source 1..21: mol_type = other DNA
agagcacctcagagagtacaa

SEQ ID NO: 126 - DNA, 18 bp, synthetic construct
  source 1..18: mol_type = other DNA
gtccttccttccctccct

SEQ ID NO: 127 - DNA, 20 bp, synthetic construct
  source 1..20: mol_type = other DNA
ctattttgtgtgctacctat

SEQ ID NO: 128 - DNA, 21 bp, synthetic construct
  source 1..21: mol_type = other DNA
caattttccattaattgaatc

SEQ ID NO: 129 - DNA, 21 bp, synthetic construct
  source 1..21: mol_type = other DNA
atataagctatcaaacagatc

SEQ ID NO: 130 - DNA, 21 bp, synthetic construct
  source 1..21: mol_type = other DNA
atttatgtcaaaaatcaaggc

SEQ ID NO: 131 - DNA, 21 bp, synthetic construct
  source 1..21: mol_type = other DNA
ctgtataactgaggtgaaagc

SEQ ID NO: 132 - DNA, 21 bp, synthetic construct
  source 1..21: mol_type = other DNA
ttcttcctatccctgtactcc

SEQ ID NO: 133 - DNA, 21 bp, synthetic construct
  source 1..21: mol_type = other DNA
ttgtgggggagacaccttaaa

SEQ ID NO: 134 - DNA, 21 bp, synthetic construct
  source 1..21: mol_type = other DNA
taaagtatgtacctactgtgg

SEQ ID NO: 135 - DNA, 21 bp, synthetic construct
  source 1..21: mol_type = other DNA
ctcaaaccccttcagattaat

SEQ ID NO: 136 - DNA, 21 bp, synthetic construct
  source 1..21: mol_type = other DNA
ccacaccaggctaagttttgt

SEQ ID NO: 137 - DNA, 21 bp, synthetic construct
  source 1..21: mol_type = other DNA
aatggtacatttggtgaggag

SEQ ID NO: 138 - DNA, 21 bp, synthetic construct
  source 1..21: mol_type = other DNA
aggctcactaagggtgattac

SEQ ID NO: 139 - protein, 8 aa, synthetic construct
DYKDDDDK

SEQ ID NO: 140 - protein, 261 aa, synthetic construct
KRPAATKKAGQAKKKKGSEGFPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 141 - protein, 499 aa, synthetic construct
MFNKSFGTPFGGGTGGFGTTSTFGQNTGFGTTSGGAFGTSAFGSSNNTGGLFGNSQTKPGGLFGTSSFSQPATSTSTGFGFGTSTGTANTLFGTASTGTSLFSSQNNAFAQNKPTGFGNFGTSTSSGGLFGTTNTTSNPFGSTSGSLFGPSSFTAAPTGTTIKFNPPTGTDTMVKAGVSTNISTKHQCITAMKEYESKSLEELRLEDYQANRKGPQNQVGAGTTTGLFGSSPATSSATGLFSSSTTNSGFAYGQNKTAFGTSTTGFGTNPGGLFGQQNQQTTSLFSKPFGQATTTQNTGFSFGNTSTIGQPSTNTMGLFGVTQASQPGGLFGTATNTSTGTAFGTGTGLFGQTNTGFGAVGSTLFGNNKLTTFGSSTTSAPSFGTTSGGLFGFGTNTSGNSIFGSKPAPGTLGTGLGAGFGTALGAGQASLFGNNQPKIGGPLGTGAFGAPGFNTTTATLGFGAPQAPVALTDPNASAAQQAVLQQHINSLTYSPFGDS

SEQ ID NO: 142 - protein, 308 aa, synthetic construct
MFNKSFGTPFGGGTGGFGTTSTFGQNTGFGTTSGGAFGTSAFGSSNNTGGLFGNSQTKPGGLFGTSSFSQPATSTSTGFGFGTSTGTANTLFGTASTGTSLFSSQNNAFAQNKPTGFGNFGTSTSSGGLFGTTNTTSNPFGSTSGSLFGPSSFTAAPTGTTIKFNPPTGTDTMVKAGVSTNISTKHQCITAMKEYESKSLEELRLEDYQANRKGPQNQVGAGTTTGLFGSSPATSSATGLFSSSTTNSGFAYGTGAFGAPGFNTTTATLGFGAPQAPVALTDPNASAAQQAVLQQHINSLTYSPFGDS

SEQ ID NO: 143 - protein, 86 aa, synthetic construct
MFNKSFGTPFGGGTGGFGTTSTFGQNTGFGTTSGGAFGTSAFGSSNNTGGLFGNSQTKPGGLFGTSSFSQPATSTSTGFGFGTSTG

SEQ ID NO: 144 - protein, 190 aa, synthetic construct
QNKTAFGTSTTGFGTNPGGLFGQQNQQTTSLFSKPFGQATTTQNTGFSFGNTSTIGQPSTNTMGLFGVTQASQPGGLFGTATNTSTGTAFGTGTGLFGQTNTGFGAVGSTLFGNNKLTTFGSSTTSAPSFGTTSGGLFGFGTNTSGNSIFGSKPAPGTLGTGLGAGFGTALGAGQASLFGNNQPKIGGPL

SEQ ID NO: 145 - protein, 281 aa, synthetic construct
MFNKSFGTPFGGGTGGFGTTSTFGQNTGFGTTSGGAFGTSAFGSSNNTGGLFGNSQTKPGGLFGTSSFSQPATSTSTGFGFGTSTGTANTLFGTASTGTSLFSSQNNAFAQNKPTGFGNFGTSTSSGGLFGTTNTTSNPFGSTSGSLFGPSSFTAAPTGTTIKFNPPTGTDTMVKAGVSTNISTKHQCITAMKEYESKSLEELRLEDYQANRKGPQNQVGAGTTTGLFGSSPATSSATGLFSSSTTNSGFAYGQNKTAFGTSTTGFGTNPGGLFGQQNQQT

SEQ ID NO: 146 - protein, 377 aa, synthetic construct
MFNKSFGTPFGGGTGGFGTTSTFGQNTGFGTTSGGAFGTSAFGSSNNTGGLFGNSQTKPGGLFGTSSFSQPATSTSTGFGFGTSTGTANTLFGTASTGTSLFSSQNNAFAQNKPTGFGNFGTSTSSGGLFGTTNTTSNPFGSTSGSLFGPSSFTAAPTGTTIKFNPPTGTDTMVKAGVSTNISTKHQCITAMKEYESKSLEELRLEDYQANRKGPQNQVGAGTTTGLFGSSPATSSATGLFSSSTTNSGFAYGQNKTAFGTSTTGFGTNPGGLFGQQNQQTTSLFSKPFGQATTTQNTGFSFGNTSTIGQPSTNTMGLFGVTQASQPGGLFGTATNTSTGTAFGTGTGLFGQTNTGFGAVGSTLFGNNKLTTFGSST

SEQ ID NO: 147 - protein, 316 aa, synthetic construct
QNKTAFGTSTTGFGTNPGGLFGQQNQQTTSLFSKPFGQATTTQNTGFSFGNTSTIGQPSTNTMGLFGVTQASQPGGLFGTATNTSTGTAFGTGTGLFGQTNTGFGAVGSTLFGNNKLTTFGSSTTSAPSFGTTSGGLFGFGTNTSGNSIFGSKPAPGTQNKTAFGTSTTGFGTNPGGLFGQQNQQTTSLFSKPFGQATTTQNTGFSFGNTSTIGQPSTNTMGLFGVTQASQPGGLFGTATNTSTGTAFGTGTGLFGQTNTGFGAVGSTLFGNNKLTTFGSSTTSAPSFGTTSGGLFGFGTNTSGNSIFGSKPAPGT

SEQ ID NO: 148 - protein, 499 aa, synthetic construct; identical to SEQ ID NO: 141

SEQ ID NO: 149 - protein, 499 aa, synthetic construct
MFNKSSGTPSGGGTGGSGTTSTSGQNTGSGTTSGGASGTSASGSSNNTGGLSGNSQTKPGGLSGTSSFSQPATSTSTGSGSGTSTGTANTLSGTASTGTSLFSSQNNAFAQNKPTGSGNSGTSTSSGGLSGTTNTTSNPSGSTSGSLSGPSSFTAAPTGTTIKFNPPTGTDTMVKAGVSTNISTKHQCITAMKEYESKSLEELRLEDYQANRKGPQNQVGAGTTTGLSGSSPATSSATGLFSSSTTNSGFAYGQNKTASGTSTTGSGTNPGGLSGQQNQQTTSLFSKPSGQATTTQNTGFSSGNTSTIGQPSTNTMGLSGVTQASQPGGLSGTATNTSTGTASGTGTGLSGQTNTGSGAVGSTLSGNNKLTTSGSSTTSAPSSGTTSGGLSGSGTNTSGNSISGSKPAPGTLGTGLGAGSGTALGAGQASLSGNNQPKIGGPLGTGASGAPGFNTTTATLGSGAPQAPVALTDPNASAAQQAVLQQHINSLTYSPSGDS

SEQ ID NO: 150 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 151 - protein, 109 aa, synthetic construct
VDREKQPSEGAFSENNAENESGGDKPPIDPNNPAANWLHARSTRKKRCPYTKHQTLELEKEFLFNMYLTRDRRYEVARLLNLTERQVKIWFQNRRMKMKKINKDRAKDE

SEQ ID NO: 152 - protein, 205 aa, synthetic construct
DDSMEEKPLKVKGKDSSEKKRKRKLEKVEQLFGEGKQKSKELKKMDKPRKKKLKLGADKSKELNKLAKKLAKEEERKKKKEKAAAAKVELVKESTEKKREKKVLDIPSKYDWSGAEESDDENAVCAAQNCQRPCKDKVDWVQCDGGCDEWFHQVCVGVSPEMAENEDYICINCAKKQGPVSPGPAPPPSFIMSYKLPMEDLKETS

SEQ ID NO: 153 - protein, 1408 aa, synthetic construct
MESTETEERSTELKTEIKEEEDQPSTSATQSSPAPGQSKKKIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDRFVYTCNECKHHVETRWHCTVCEDYDLCITCYNTKNHDHKMEKLGLGLDDESNNQQAAATQSPGDSRRLSIQRCIQSLVHACQCRNANCSLPSCQKMKRVVQHTKGCKRKTNGGCPICKQLIALCCYHAKHCQENKCPVPFCLNIKQKLRQQQLQHRLQQAQMLRRRMASMQRTGVVGQQQGLPSPTPATPTTPTGQQPTTPQTPQPTSQPQPTPPNSMPPYLPRTQAAGPVSQGKAAGQVTPPTPPQTAQPPLPGPPPAAVEMAMQIQRAAETQRQMAHVQIFQRPIQHQMPPMTPMAPMGMNPPPMTRGPSGHLEPGMGPTGMQQQPPWSQGGLPQPQQLQSGMPRPAMMSVAQHGQPLNMAPQPGLGQVGISPLKPGTVSQQALQNLLRTLRSPSSPLQQQQVLSILHANPQLLAAFIKQRAAKYANSNPQPIPGQPGMPQGQPGLQPPTMPGQQGVHSNPAMQNMNPMQAGVQRAGLPQQQPQQQLQPPMGGMSPQAQQMNMNHNTMPSQFRDILRRQQMMQQQQQQGAGPGIGPGMANHNQFQQPQGVGYPPQQQQRMQHHMQQMQQGNMGQIGQLPQALGAEAGASLQAYQQRLLQQQMGSPVQPNPMSPQQHMLPNQAQSPHLQGQQIPNSLSNQVRSPQPVPSPRPQSQPPHSSPSPRMQPQPSPHHVSPQTSSPHPGLVAAQANPMEQGHFASPDQNSMLSQLASNPGMANLHGASATDLGLSTDNSDLNSNLSQSTLDIH

SEQ ID NO: 154 - protein, 236 aa, synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

(19) United States
(12) Patent Application Publication
(10) Pub. No.: US 2026/0337587 A1
(43) Pub. Date: Oct. 29, 2026

(54) HIGHLY EFFICIENT SELF-ASSEMBLING PROGRAMMABLE GENOME AND EPIGENOME EDITING TOOLS

(71) Applicant: William Marsh Rice University, Houston, TX (US)

(72) Inventors: Isaac B. HILTON, Houston, TX (US); Jing LI, Houston, TX (US); Shizhe LIU, Houston, TX (US)

(73) Assignee: William Marsh Rice University, Houston, TX (US)

(21) Appl. No.: 19/163,828

(22) PCT Filed: Mar. 8, 2024
(86) PCT No.: PCT/US2024/019102

§ 371 Date: Sep. 9, 2025

Related U.S. Application Data
(60) Provisional application No. 63/489,545, filed on Mar. 10, 2023.

(51) Int. Cl. C12N 9/22; A61K 48/00; C12N 15/10; C12N 15/113; C12N 15/52; C12N 15/62

(52) U.S. Cl. CPC C12N 9/226; A61K 48/00; C12N 15/102; C12N 15/113; C12N 15/52; C12N 15/62; C12N 2310/20

(57) ABSTRACT

The present disclosure is directed to methods that universally and robustly enhance the genome and epigenome editing efficacies of CRISPR-based technologies by concentrating the DNA/chromatin effectors at the targeted gene regulatory loci using novel engineered proteins and molecular fusions. The fusion of Cas9 proteins to FG domains significantly enhances self-assembly and high concentration of CRISP-Cas9 machinery in the nucleus and specific loci therein.

### PRIORITY CLAIM

This application claims benefit of priority to U.S. Provisional Application Ser. No. 63/489,545, filed Mar. 10, 2023, the entire contents of which are hereby incorporated by reference.

### STATEMENT REGARDING FEDERALLY SPONSORED RESEARCH AND DEVELOPMENT

This invention was made with government support under R35GM143532 and R56HG012206 awarded by the National Institutes of Health. The government has certain rights in the invention.

### REFERENCE TO A SEQUENCE LISTING

This application contains a Sequence Listing XML, which has been submitted electronically and is hereby incorporated by reference in its entirety. Said XML Sequence Listing, created on Mar. 6, 2024, is named RICEP0126WO.xml and is 142,841 bytes in size.

### BACKGROUND

### 1. Field of the Disclosure

The present disclosure relates generally to the fields of medicine, molecular biology, gene regulation and genetic therapies. More particularly, the disclosure relates to improved methods of genome and epigenome editing efficacies.

### 2. Background

Genome and epigenome editing technologies have matured to enable altering DNA sequences, transcription, and/or chromatin structure in a site-directed way. Therefore, they now decidedly hold great promise to prevent, study, and even cure human diseases. However, their applications in human therapeutics can be limited by low efficiencies, and improved methods are thereof needed.

NUP98 is a conserved component of the nuclear pore complex (NPC) in eukaryotes that works in concert with other NPC proteins to facilitate bidirectional nuclear transport of macromolecules. However, genomic translocations and inversions can result in aberrant fusions between the N-terminal NUP98 phenylalanine-glycine (FG) repeat domain and other DNA- or chromatin binding domains which consequently drive the NUP98 FG repeat domain off-pore (Franks & Hetzer, 2013). This off-pore activity is linked to a series of pathological processes including the nuclear condensation of NUP98 FG repeats (Ahn et al., 2021; Chandra et al., 2022; Terlecki-Zaniewicz et al., 2021), inappropriate chromatin looping (Ahn et al., 2021), and dysregulated gene expression (Chandra et al., 2022; Terlecki-Zaniewicz et al., 2021). Unfortunately, these abnormal processes converge to drive leukemic transformation and a subset of clinically intractable hematologic cancers (Chandra et al., 2022; Gough et al., 2011). Despite this clinical importance, it remains incompletely understood how the NUP98 FG repeat domain dysregulates gene expression and in turn drives oncogenesis. This gap in knowledge directly limits the mechanistic understanding of pathological NUP98 fusion proteins as well as the development of new therapeutic options.

Understanding how the NUP98 FG repeat domain drives hematologic cancers is further confounded by the extensive diversity of oncogenic NUP98 fusions observed in patients. For example, NUP98 fusion proteins harboring as few as 19 to as many as 39 FG repeats have been observed in human cancers (Gough et al., 2011; Arai et al., 1997; Hussey et al., 2001; Kasper et al., 1999). This range of FG repeats arises from different NUP98 isoforms (Kasper et al., 1999) and from differing chromosomal break points within the NUP98 coding region (Arai et al., 1997; Hussey et al., 2001). Additionally, at least 30 different and unique NUP98 fusion partners have been identified clinically (Gough et al., 2011; Michmerhuizen et al., 2020) that further diversify the landscape of oncogenic NUP98 fusions. These fusion partners are functionally varied and encompass a wide array of different DNA binding and epigenetic regulatory domains-such as homeodomains, PHD, and SET domains (Gough et al., 2011; Michmerhuizen et al., 2020). This multilayered diversity among oncogenic NUP98 fusions has raised the question as to whether highly heterogenous NUP98 fusions mediate discrete pathological cellular processes or if NUP98 fusions drive oncogenesis through a conserved mechanistic route. A better understanding of the functionality of NUP98 fusions would thus improve therapeutic interventions.

### SUMMARY

Thus, in accordance with the present disclosure, there is provided a fusion protein comprising (a) a phenylalanine/glycine (FG) domain comprising repeating patterns of phenylalanine (F) and glycine (G) and (b) a Cas9 protein. The FG domain does not bind to RNA export factor 1.

The fusion protein may further comprise an effector protein. The effector protein may be a transcription activator, a transcription co-factor, a transcription repressor, a histone acetylase, or a DNA demethylase. The effector protein may be p300, KRAB, VP64, TET1 (e.g., dTET1), eN3x, HP1a-d, BRD4, OCT4, MED14, or MED23.

The Cas9 protein may be a CRISPR-based transcriptional activator, a CRISPR-based transcriptional repressor, or a CRISPR-based epigenome editor, a CRIPSR-based nucleases, a CRIPSR-base editor, a CRIPSR-based prime editor, a CRIPSR-based transposase, a dCas9-modifier of DNA methylation, a dCa9-modifier of histone methylation, or a dCas9-modifier of histone phosphorylation.

The FG domain may comprise 10 to 50 F/G repeats, 18 F/G repeats, 21 F/G repeats, 32 F/G repeats, or 39 F/G repeats. The FG domain may comprise or consist of an amino acid sequence at least 95%, at least 96%, at least 97%, at least 98%, at least 99%, or 100% identical to the amino acid sequence of SEQ ID NO: 144 or 147.

There is provided a pharmaceutical composition comprising a fusion protein provided herein. There is provided a nucleic acid encoding a fusion protein provided herein. There is provided a cell comprising a fusion protein a nucleic acid provided herein.

There is provided a method of transporting a Cas9 protein into a cell nucleus comprising introducing into a cell a fusion protein or a nucleic acid provided herein. There is provided a method of modifying the phenotype of a cell comprising introducing a fusion protein or a nucleic acid provided herein, into the cell, along with a gRNA. The introduction of the fusion protein and gRNA into the cell may cause targeted modification of the epigenome of the cell. The introduction of the fusion protein and gRNA into the cell may cause transcription activation or repression of a target gene. The introduction of the fusion protein and gRNA into the cell may cause targeted histone acetylation or DNA demethylation.

There is provided a kit comprising a fusion protein or a nucleic acid provided herein, optionally further comprising a gRNA.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-F: NUP98 FG domains activate gene expression when localized to human promoters. (FIG. 1A) Expression and cellular distribution of EGFP, NUP98a-EGFP and NUP98b-EGFP in HEK293T cells ~20 hours post-transfection. White dotted lines encircle nuclei and red scale bar=10 μm. (FIGS. 1B-C). Quantification of nuclear (FIG. 1B) and condensed (FIG. 1C) EGFP fractions in HEK293T cells ~20 hours post-transfection of EGFP control, NUP98a-EGFP or NUP98b-EGFP. Asterisks (*) indicate P<0.05 via two-tailed student's t-test. n=10 randomly selected cells; error bars; s.e.m. (FIG. 1D) Schematics of dCas9, dCas9 fused to the NUP98a FG domain (dCas9-NUP98a), and dCas9 fused to the NUP98b FG domain (dCas9-NUP98b). Vertical stripes in NUP98 segments indicate the relative locations of 39 and 21 FG repeats for NUP98a and NUP98b, respectively. (FIG. 1E) RT-qPCR for IL1RN, OCT4, or HBG1 72 hours post-transfection of dCas9, dCas9-NUP98a, or dCas9-NUP98b and 4 corresponding promoter-targeting gRNAs. Asterisks (*) indicate P<0.05 via two-tailed student's t-test. (FIG. 1F) mRNA levels of IL1RN, OCT4, or HBG1 were measured by RT-qPCR 72 hours after co-transfection of dCas9-NUP98a and corresponding promoter-targeting single gRNA or indicated gRNA combinations. An mRNA dose-response curve was performed with the number of gRNA targeted sites ranging from 0 to 4. n=3 independent experiments for FIGS. 1C-D; error bars; s.e.m.

FIGS. 2A-D: The ability of dCas9-NUP98 to activate human promoters is linked to its ability to phase separate in cell nuclei. (FIG. 2A) Expression and cellular localization of dCas9-EGFP, dCas9-NUP98a-EGFP, dCas9-NUP98b-EGFP, or dCas9-p300-EGFP ~20 hours post-transfection in HEK293T cells before and 1 minute after treatment with 10% 1,6-hexanediol. Dotted lines encircle nuclei and red scale bar=10 μm. (FIGS. 2B-C) Quantification of condensed (FIG. 2B) and nuclear (FIG. 2C) EGFP fractions in HEK293T cell ~20 hours post-transfection of dCas9-EGFP, dCas9-NUP98a-EGFP, dCas9-NUP98b-EGFP, or dCas9-p300-EGFP. Asterisks (*) indicate P<0.05 via two-tailed student's t-test. n=10 randomly selected cells; error bars; s.e.m. (FIG. 2C) RT-qPCR for IL1RN (left), OCT4 (middle), or HBG1 (right) 72 hours post-transfection of dCas9 or indicated dCas9-NUP98 fusions and corresponding gRNAs in the absence or presence of 0.4% 1,6-hexanediol. (FIG. 2D) RT-qPCR for IL1RN 72 hours post-transfection of dCas9-p300 and 4 corresponding gRNAs in the absence or presence of 0.4% 1,6-hexanediol. Asterisks (*) indicate P<0.05 via two-tailed student's t-test. n=3 independent experiments for FIGS. 2C-D; error bars; s.e.m. ns; not significant.

FIGS. 3A-D: NUP98 FG repeat numbers correlate with phase separation behavior and gene activation potency. (FIG. 3A) Expression and cellular localization of EGFP-tagged NUP98 FG or phenylalanine to serine (F/S) mutant and dCas9-NUP98 FG (or F/S mutant) repeat variants (top and bottom, respectively) in HEK293T cells ~20 hours post-transfection. Dotted lines encircle nuclei and red scale bar=10 μm. (FIG. 3B) Quantification of nuclear (left) and condensed (right) EGFP fractions in HEK293T cells ~20 hours post-transfection of EGFP tagged NUP98 FG (or F/S mutant) repeat variants. n=10 randomly selected cells; error bars, s.e.m. (FIG. 3C) Quantification of nuclear (left) and condensed (right) EGFP fractions in HEK293T cells ~20 hours post-transfection of EGFP tagged dCas9-NUP98 FG (or F/S mutant) repeat variants. n=10 randomly selected cells; error bars, s.e.m. (FIG. 3C) RT-qPCR for IL1RN (left), OCT4 (middle), or HBG1 (right) mRNA level ~72 hours post-transfection of dCas9-EGFP or indicated dCas9-FG repeat variants and corresponding gRNAs. n=3 independent experiments; error bars; s.e.m.

FIGS. 4A-E: Oncogenic fusion partners control the genomic localization of NUP98 FG repeats. (FIGS. 4A-B) Fluorescence microscopy of NUP98a control, NUP98a-HOXA9, NUP98a-KDM5A, NUP98b control, NUP98b-HOXA9 or NUP98b-KDM5A (FIG. 4A) or respective constructs directly fused to the C-terminus of dCas9 (FIG. 4B) in HEK293T cells ~20 hours post-transfection. All plasmid constructs harbor C-terminal EGFP fusions. Dotted lines encircle nuclei and red scale bar=10 μm. (FIG. 4C) Genome-wide binding analysis of NUP98a, NUP98a-HOXA9, NUP98a-KDM5A, or dCas9-NUP98a co-transfected with four IL1RN promoter-targeting gRNAs in HEK293T ~72 hours post-transfection. Pink dots indicate binding above a False Discovery Rate (FDR) of 0.05. (FIGS. 4D-E) RT-qPCR for IL1RN (left), OCT4 (middle), or HBG1 (right) ~72 hours post-transfection of dCas9 or indicated dCas9-NUP98a fusions (FIG. 4D) or indicated dCas9-NUP98b fusions (FIG. 4E) with corresponding gRNAs. n=3 independent experiments for FIGS. 4D-E; error bars; s.e.m.

FIGS. 5A-F: NUP98a-HOXA9 and NUP98a-KDM5A display distinct genomic binding profiles. (FIG. 5A) Genomic distribution of NUP98a-HOXA9 and NUP98a-KDM5A relative to all human transcription start sites (TSSs) ~72 hours post-transfection in HEK293T cells. (FIGS. 5B-C) The distribution of NUP98a-HOXA9 and NUP98a-KDM5A binding with respect to genomic annotations (FIG. 5B) and distance to TSS (FIG. 5C) using ChIPseeker. (FIG. 5D) The genomic regions encompassing the MLLT3 and MEIS1 genes are shown along with ChIP-seq signals from wildtype HEK293T cells (black), NUP98a-HOXA9 (orange) and NUP98a-KDM5A (purple) transfected HEK293T cells. Genomic coordinates are based upon GRCh38/hg38. gRNAs are indicated with red bars and 3C-qPCR primers are indicated with red arrows. Chromatin contacts detected via 3C-qPCR are labelled as blue arced lines. (FIG. 5E) RT-qPCR for MLLT3 (left) and MEIS1 (right) mRNA 72 hours post-transfection of dCas9-NUP98a and corresponding gRNAs. (FIG. 5F) Relative contact (using 3C-qPCR) between the MLLT3 or MEIS1 promoters and NUP98a-HOXA9 bound introns 2-weeks post-transduction with EGFP, NUP98a-HOXA9-EGFP and NUP98a-KDM5A-EGFP in HEK293T. For FIGS. 5E and 5F, asterisks (*) indicate P<0.05 via two-tailed student's t-test. n=3 independent experiments; error bars; s.e.m.

FIGS. 6A-F: NUP98-HOXA9 and NUP98-KDM5A drive a shared leukemogenic transcriptional program in primary human hematopoietic stem cells. (FIG. 6A) Expression of EGFP control, NUP98a-HOXA9 and NUP98a-KDM5A ~72 hours post lentiviral transduction in primary human hematopoietic stem cells (HSCs). All NUP98-encoding lentiviral constructs also harbor C-terminal EGFP fusions. Dotted lines encircle nuclei and red scale bar=10 μm. (FIG. 6B) PCA analysis of HSC transcriptomes after transduction with EGFP, NUP98a-HOXA9-EGFP, or NUP98a-KDM5A-EGFP. (FIG. 6C) Venn diagram showing the number and overlap of significantly upregulated (top) and downregulated (bottom) genes identified using RNA-seq in the indicated conditions ~14 days post lentiviral transduction. (FIG. 6D) KEGG analysis of all upregulated genes in human HSCs transduced by NUP98a-HOXA9-EGFP or NUP98a-KDM5A-EGFP. (FIG. 6E) Heatmap showing differentially expressed genes in human HSCs transduced with EGFP, NUP98a-HOXA9-EGFP and NUP98a-KDM5A-EGFP. (FIG. 6F) KEGG analysis of all downregulated genes in human HSCs transduced by NUP98a-HOXA9-EGFP or NUP98a-KDM5A-EGFP.

FIG. 7. NUP98 FG domains fused to dCas9 nonlinearly activate gene expression when precisely targeted to human promoters. (FIG. 7A) Schematics of EGFP, NUP98a-EGFP, and NUP98b-EGFP. Vertical stripes in the NUP98a/b segments indicate the approximate locations of 39 and 21 FG repeats for NUP98a and NUP98b, respectively. (FIG. 7B) Experimental workflow used to quantify miniCMV promoter activation capabilities of dCas9-NUP98 fusions using a HEK293T reporter cell line. Relative EGFP measured by flow cytometry 72 hours post-transfection of indicated dCas9 or dCas9-NUP98 expression vectors and miniCMV promoter targeting gRNA in HEK293T reporter cell line. EGFP intensity was normalized to dCas9 control. (FIG. 7C) mRNA levels of IL1RN (left), OCT4 (middle), or HBG1 (right) measured by RT-qPCR 72 hours after co-transfection of dCas9-NUP98a and corresponding promoter-targeting single gRNAs or indicated gRNA combinations. (FIG. 7D) Relative mRNA levels for IL1RN, OCT4, and HBG1 (plotted on log-linear scales) and the number of gRNA targeted sites ranging from 0 to 4. R squared (R2) values are shown. (FIG. 7E) mRNA levels of IL1RN measured by RT-qPCR 72 hours after co-transfection of dCas9-p300 and corresponding single gRNA or indicated gRNA combinations. (FIG. 7F) IL1RN mRNA levels induced by dCas9-p300 with increasing numbers of gRNA targeted sites (1 to 4) plotted in a linear-linear scale (left) and in log-linear scale (right). R squared (R2) values are shown. Asterisks (*) indicate P<0.05 via two-tailed student's t-test. n=3 independent experiments for FIGS. 7C-F; error bars; s.e.m.

FIGS. 8A-C. Schematic depictions of NUP98 FG domain variants fused to EGFP and/or dCas9 and EGFP. (FIG. 8A) Schematics of dCas9-EGFP, dCas9-NUP98a-EGFP, dCas9-NUP98b-EGFP, and dCas9-p300-EGFP. Vertical stripes in NUP98a/b segments indicate the approximate locations of 39 and 21 FG repeats for NUP98a and NUP98b, respectively. (FIGS. 8B-C) Schematics of EGFP, and FG-EGFP variants (panel b), and dCas9-EGFP and dCas9-FG-EGFP variants (panel c). Vertical stripes indicate the approximate locations of NUP98 FG repeats.

FIGS. 9A-D. dCas9-NUP98a and dCas9-NUP98b interact with scaffolding and gene-regulatory proteins in human cells. (FIGS. 9A-B) Volcano plots comparing the interacting proteins of dCas9-NUP98a (FIG. 9A) and dCas9-NUP98b (FIG. 9B) to dCas9 control in HEK293T cells 48 hours post-transfection. Red and blue dots indicate proteins significantly (P<0.001) enriched or depleted, respectively in dCas9-NUP98a or dCas9-NUP98b compared to dCas9 control. (FIG. 9C) Volcano plots comparing the interacting proteins of dCas9-NUP98a to dCas9-NUP98b in HEK293T cells 48 hours post-transfection. Red and blue dots indicate proteins significantly (P<0.001) enriched or depleted, respectively in dCas9-NUP98a compared to dCas9-NUP98b. (FIG. 9D) All significantly (P<0.001) enriched proteins that interact with dCas9-NUP98a and dCas9-NUP98b compared to dCas9 control are schematically depicted, along with respective functional classes. Experiments based on two independent replicates.

FIGS. 10A-E. The transcriptional coactivator p300 interacts with FG domains from NUP98. (FIG. 10A) Schematics of EGFP control, NUP98a-EGFP, NUP98b-EGFP, p300-mCherry, and mCherry control. (FIG. 10B) Co-expression of NUP98a/b-EGFP and p300-mCherry. Red scale bar=10 μm. (FIG. 10C) Co-expression of FG-EGFP fusion variants and p300-mCherry. Red scale bar=10 μm. (FIG. 10D) Relative enrichment of H3K27ac at IL1RN (left), OCT4 (middle), and HBG1 (right) promoters 72 hours post-transfection with dCas9 or indicated dCas9-NUP98 fusions and corresponding gRNAs. The genomic region encompassing the promoter of each respective gene is shown along with the associated protomer-targeting gRNAs used. Genomic coordinates are based upon GRCh38/hg38. Respective gRNA locations are indicated in red and CUT & RUN qPCR amplicons in gray. (FIG. 10E) RT-qPCR for IL1RN (left), OCT4 (middle), and HBG1 (right) mRNA levels 72 hours post-transfection of dCas9 or indicated dCas9-NUP98 fusion proteins and corresponding gRNAs in the presence of DMSO or the CBP/p300 inhibitor A485. 20 μM of A485 or an equal volume of DMSO was added to cells 6 hours post-transfection. For panel d and e, Asterisks (*) indicate P<0.05 via two-tailed student's t-test. n=3 independent experiments; error bars; s.e.m.

FIGS. 11A-E. DNA binding profiles of NUP98 FG domains fused to dCas9 and oncogenic fusion partner. (FIGS. 11A-B) Schematics of NUP98a/b-EGFP, NUP98a/b-HOXA9-EGFP, and NUP98a/b-KDM5A-EGFP (FIG. 11A) or dCas9-NUP98a/b-EGFP, dCas9-NUP98a/b-HOXA9-EGFP, and dCas9-NUP98a/b-KDM5A-EGFP (FIG. 11B). (FIG. 11C) Quantification of nuclear EGFP fractions in HEK293T cells ~20 hours post-transfection of NUP98b-EGFP, HOXA9-NUP98b-EGFP, KDM5A-NUP98b-EGFP, dCas9-NUP98b-EGFP, dCas9-HOXA9-NUP98b-EGFP or dCas9-KDM5A-NUP98b-EGFP. Asterisks (*) indicate P<0.05 via two-tailed student's t-test. n=10 randomly selected cells; error bars; s.e.m. (FIG. 11D) Genome-wide binding analysis of dCas9-NUP98a-HOXA9 or dCas9-NUP98a-KDM5A co-transfected with four IL1RN promoter-targeting gRNAs in HEK293T 72 hours post-transfection. Pink dots indicate a False Discovery Rate (FDR)<0.050 relative to wt HEK293T cells. (FIG. 11E) The genomic region (based upon GRCh38/hg38) encompassing the IL1RN promoter is shown along with gRNAs (in red) used for targeting.

FIGS. 12A-F. DNA binding landscapes of NUP98a-HOXA9 and NUP98a-KDM5A in human cells. (FIG. 12A) Genome-wide binding of NUP98a-HOXA9 compared to NUP98a-KDM5A in HEK293T ~72 hours post-transfection. Pink dots indicate a False Discovery Rate (FDR)<0.050. (FIG. 12B) Pie charts showing the genomic distributions of NUP98a-HOXA9 (left) and NUP98a-KDM5A (right) using ChIP-seeker and corresponding annotations. (FIG. 12C) The respective numbers of genes near NUP98a-HOXA9 or NUP98a-KDM5A binding sites, respectively, ~72 hours post-transfection in HEK293T cells. (FIG. 12D) KEGG analysis of all genes identified in FIG. 12C. (FIG. 12E) The genomic regions encompassing the FUT8, HMGA2, PBX3, SUPT3H, RUNX2, HOXA9 and ZBTB16 genes within the transcriptional dysregulation in cancer KEGG pathway are shown. ChIP-seq signal from wildtype HEK293T cells (black), NUP98a-HOXA9 (orange) and NUP98a-KDM5A (purple) transfected HEK293T are shown beneath each locus. Genomic coordinates are based upon GRCh38/hg38. n=2 independent ChIP-seq experiments for each condition. (FIG. 12F) mRNA levels of HBG1 (left), OCT4 (middle and right) measured by RT-qPCR 72 hours after co-transfection of dCas9-NUP98a and indicated gRNAs targeting on the HS2 enhancers of HBG1 (HS2, left), distal enhancer of OCT4 (DE; middle) and proximal enhancer of PCT4 (PE, right). n=3 independent experiments.

FIGS. 13A-E. Oncogenic NUP98 fusion proteins dysregulate gene expression patterns in primary human HSCs. (FIG. 13A) Expression of EGFP control, NUP98b-HOXA9 and NUP98b-KDM5A 72 hours post lentiviral transduction in primary human hematopoietic stem cells (HSCs). All NUP98-encoding lentiviral constructs also harbor C-terminal EGFP fusions. Dotted lines encircle nuclei and red scale bar=10 μm. (FIG. 13B) mRNA levels of NUP98-HOXA9 (left) and NUP98-KDM5A (right) fusions were measured by RT-qPCR 2 weeks post-transduction. (FIG. 13C) PCA analysis of the transcriptomes of human HSCs transduced with EGFP, NUP98a-HOXA9-EGFP, NUP98a-KDM5A-EGFP, NUP98b-HOXA9-EGFP, or NUP98b-KDM5A-EGFP 2 weeks post-transduction. (FIG. 13D) Venn diagram showing overlap among significantly upregulated (top) and downregulated (bottom) genes identified using bulk RNA-seq in the indicated conditions 14 days post lentiviral transduction. ah, NUP98a-HOXA9-EGFP; ak, NUP98a-KDM5A-EGFP; bh, NUP98b-HOXA9-EGFP; bk, NUP98b-KDM5A-EGFP. (FIG. 13E) Volcano plots showing differentially expressed genes in primary human HSCs stably transduced with NUP98a/b-HOXA9-EGFP or NUP98a/b-KDM5A-EGFP relative to cells transduced with EGFP. Red and blue dots indicate significantly (Padj<0.01) upregulated and downregulated genes, respectively.

FIG. 14. 8A-C. Oncogenic NUP98 fusion proteins drive a shared leukemic transcriptional program in primary human HSCs. (FIG. 13A) KEGG analysis of all significantly (Padj<0.01) upregulated genes identified using bulk RNA-seq data from primary HSCs transduced with the indicated NUP98 fusions compared to EGFP transduced HSCs. (FIG. 13B) Heatmap of all differential genes identified though RNA-seq in primary HSCs across the indicated transduction conditions. (FIG. 13C) KEGG analysis of all significantly (Padj<0.01) downregulated genes identified using bulk RNA-seq data from primary HSCs transduced with the indicated NUP98 fusions compared to EGFP transduced HSCs.

FIG. 15. Amino acid sequences of protein domains used.

FIGS. 16A-E. Engineering a synthetic FG domain that does not bind to RAE1. (FIG. 16A) a model of traditional and self-assembling CRISPR tools. (FIG. 16B) Mass spec analysis of the interactome of dCas9-NUP98a, dCas9-NUP98b and dCas9-18FG. (FIG. 16C) Schematics of dCas9 fused to the endogenous NUP98a FG domain (dCas9-NUP98a), dCas9 fused to the endogenous NUP98b FG domain (dCas9-NUP98b) and dCas9 fused to the synthetic 18 FG domain (dCas9-18FG) and dCas9 fused to the synthetic 32 FG domain (dCas9-32FG). Vertical stripes in NUP98 segments indicate the relative locations of FG repeats. (FIG. 16D) Expression and cellular distribution of dCas9-EGFP, dCas9-18FG-EGFP and dCas9-32FG-EGFP in HEK293T cells ~20 hours post-transfection. red scale bar=10 μm. (FIG. 16E) mRNA levels of IL1RN, OCT4, or HBG1 were measured by RT-qPCR 72 hours after co-transfection of indicated dCas9-fusions and corresponding promoter-targeting gRNA combinations.

FIGS. 17A-C. Engineering new CRISPRa tools with synthetic FG domains. (FIG. 17A) Schematics of synthetic FG domains fused to the VP64-dCas9, eN3x-dCas9 and 3x9aa-dCas9. (FIG. 17B) Expression and cellular distribution of VP64-dCas9-EGFP, VP64-dCas9-18FG-EGFP, VP64-dCas9-32FG-EGFP, eN3x-dCas9-EGFP, eN3x-dCas9-18FG-EGFP, eN3x-dCas9-32FG-EGFP, 3x9aa-dCas9-EGFP, 3x9aa-dCas9-18FG-EGFP and 3x9aa-dCas9-32FG-EGFP in HEK293T cells ~20 hours post-transfection. red scale bar=10 μm. (FIG. 17C) mRNA levels of IL1RN, OCT4, HBG1 or MYOD were measured by RT-qPCR 72 hours after co-transfection of indicated dCas9-fusions and corresponding promoter-targeting gRNA combinations.

FIGS. 18A-C. Engineering new CRISPRi tools with synthetic FG domains. (FIG. 18A) Schematics of synthetic FG domains fused to KRAB-dCas9 and HP1a-dCas9. (FIG. 18B) Expression and cellular distribution of KRAB-dCas9-EGFP, KRAB-dCas9-18FG-EGFP, KRAB-dCas9-32FG-EGFP, HP1a-dCas9-EGFP, HP1a-dCas9-18FG-EGFP, HP1a-dCas9-32FG-EGFP in HEK293T cells ~20 hours post-transfection. red scale bar=10 μm. (FIG. 18C) mRNA levels of IL1RN, OCT4, HBG1 or MYOD were measured by RT-qPCR 72 hours after co-transfection of dCas9-fusions and corresponding promoter-targeting single gRNA or indicated gRNA combinations.

FIGS. 19A-C. Synthetic FG domains manipulate the transcriptional condensates. (FIG. 19A) Schematics of synthetic FG domains fused to the OCT4-dCas9 and BRD4-dCas9. (FIG. 19B) Expression and cellular distribution of OCT4-dCas9-EGFP, OCT4-dCas9-18FG-EGFP, OCT4-dCas9-32FG-EGFP, BRD4-dCas9-EGFP, BRD4-dCas9-18FG-EGFP, BRD4-dCas9-32FG-EGFP in HEK293T cells ~20 hours post-transfection. red scale bar=10 μm. (FIG. 19C) mRNA levels of IL1RN, MYOD or HBG1 were measured by RT-qPCR 72 hours after co-transfection of dCas9-fusions and corresponding promoter-targeting gRNA combinations.

FIGS. 20A-C. Synthetic FG domains manipulate the transcriptional condensates. (FIG. 20A) Schematics of synthetic FG domains fused to the MED14-dCas9 and MED23-dCas9. (FIG. 20B) Expression and cellular distribution of MED14-dCas9-EGFP, MED14-dCas9-18FG-EGFP, MED14-dCas9-32FG-EGFP, MED23-dCas9-EGFP, MED23-dCas9-18FG-EGFP, MED23-dCas9-32FG-EGFP in HEK293T cells ~20 hours post-transfection. red scale bar=10 μm. (FIG. 20C) mRNA levels of IL1RN, MYOD or HBG1 were measured by RT-qPCR 72 hours after co-transfection of dCas9-fusions and corresponding promoter-targeting gRNA combinations.

FIGS. 21A-D. Synthetic FG domains assist histone modification. (FIG. 21A) Schematics of synthetic FG domains fused to p300-dCas9 and FLp300-dCas9. (FIG. 21B) Expression and cellular distribution of p300-dCas9-EGFP, p300-dCas9-18FG-EGFP, p300-dCas9-32FG-EGFP, FLp300-dCas9-EGFP, FLp300-dCas9-18FG-EGFP, FLp300-dCas9-32FG-EGFP in HEK293T cells ~20 hours post-transfection. red scale bar=10 μm. (FIG. 21C) mRNA levels of IL1RN or HBG1 were measured by RT-qPCR 72 hours after co-transfection of dCas9-fusions and corresponding promoter-targeting gRNA combinations. (FIG. 21D) Relative enrichment of H3K27ac at IL1RN (left) and HBG1 (right) promoters 72 hours post-transfection with indicated dCas9-fusions and corresponding gRNAs.

FIGS. 22A-C. Synthetic FG domains assist DNA demethylation. (FIG. 22A) Schematics of synthetic FG domains fused to the TET1-dCas9 and dTET1-dCas9. (FIG. 22B) Expression and cellular distribution of TET1-dCas9-EGFP, TET1-dCas9-18FG-EGFP, TET1-dCas9-32FG-EGFP, dTET1-dCas9-EGFP, dTET1-dCas9-18FG-EGFP, dTET1-dCas9-32FG-EGFP in HEK293T cells ~20 hours post-transfection. red scale bar=10 μm. (FIG. 22C) mRNA levels of BRCA1, IL1RN or MYOD were measured by RT-qPCR 72 hours after co-transfection of dCas9-fusions and corresponding promoter-targeting gRNA combinations.

### DESCRIPTION OF ILLUSTRATIVE EMBODIMENTS

In this disclosure, the inventors describe a technology that universally and robustly enhances the genome and epigenome editing efficacies of CRISPR-based technologies by concentrating the DNA/chromatin effectors at the targeted gene regulatory loci using novel engineered proteins and molecular fusions. More specifically, using precision targeting of different FG repeat compositions via deactivated CRISPR/Cas9 (dCas9), the inventors demonstrate that a threshold of FG repeats is sufficient to stimulate gene expression from endogenous human promoters. They show that this transactivation acts nonlinearly, is governed by the numbers of FG repeats, binding site frequencies, and is dependent upon the ability of FG repeats to catalyze phase-separated biomolecular condensation in cell nuclei. They also generated genome-wide evidence that representatives from two major classes of oncogenic FG fusion proteins—DNA binding (NUP98-HOXA9) and chromatin modifying (NUP98-KDM5A)—have non-overlapping profiles of engagement with the human genome. Despite these discrete enrichment patterns, the inventors find that each fusion directly activates a conserved core set of leukemogenic factors in human primary hemopoietic stem cells (HSCs). Collectively, these studies reveal that FG repeats stimulate aberrant biomolecular condensation and gene expression in human cell nuclei and indicate that the localization of these aberrant behaviors to key segments of the human genome underlies the initiation of oncogenic gene expression programs. These mechanistic insights could be useful for the development of new therapeutic options for these otherwise untreatable hematologic cancers.

These and other aspects of the disclosure are described in detail below.

### I. FG DOMAINS

Nuclear pore complexes (NPCs) facilitate transport mediated by shuttling nuclear transport receptors (NTRs), while keeping nuclear and cytoplasmic contents separated. NPCs contain the intrinsically disordered FG domains (e.g., Nup98). FG domains are made up by a repeating pattern of two amino acids, phenylalanine (‘F’) and glycine (‘G’).

Nup98 is signal-mediated nuclear import and export proceed through the nuclear pore complex (NPC), which is composed of approximately 30 unique proteins collectively known as nucleoporins. The 98 kD nucleoporin is generated through a biogenesis pathway that involves synthesis and proteolytic cleavage of a 186 kD precursor protein. This cleavage results in the 98 kD nucleoporin as well as a 96 kD nucleoporin, both of which are localized to the nucleoplasmic side of the NPC. Rat studies show that the 98 kD nucleoporin functions as one of several docking site nucleoporins of transport substrates. The human gene has been shown to fuse to several genes following chromosome translocations in acute myelogenous leukemia (AML) and T-cell acute lymphocytic leukemia (T-ALL). This gene is one of several genes located in the imprinted gene domain of 11p15.5, an important tumor-suppressor gene region. Alterations in this region have been associated with the Beckwith-Wiedemann syndrome, Wilms tumor, rhabdomyosarcoma, adrenocortical carcinoma, and lung, ovarian, and breast cancer. Alternative splicing of this gene results in several transcript variants; however, not all variants have been fully described. Nup98 has been shown to interact with CREB-binding protein, KPNB1, Nup88, RAE1 and TNPO2.

Schmidt and Gorlich (2015) showed Nup98 FG domains occur in mammals, lancelets, insects, nematodes, fungi, plants, amoebas, ciliates, and excavate and spontaneously and rapidly phase-separate from dilute (submicromolar) aqueous solutions into ‘FG particles.’ All Nup98 FG phases rejected inert macromolecules and allow far larger NTR cargo complexes to rapidly enter. They even recapitulated observation that large cargo-domains counteract NPC passage of NTR·cargo complexes. This exquisite sorting selectivity and strong intrinsic assembly indicates that Nup98 FG can form in authentic NPCs and cam account for the permeability properties of the pore.

### II. CRISPR SYSTEMS

### A. CRISPRs

CRISPRs (clustered regularly interspaced short palindromic repeats) are DNA loci containing short repetitions of base sequences. Each repetition is followed by short segments of “spacer DNA” from previous exposures to a virus. CRISPRs are found in approximately 40% of sequenced eubacteria genomes and 90% of sequenced archaea. CRISPRs are often associated with Cas genes that code for proteins related to CRISPRs. The CRISPR/Cas system is a prokaryotic immune system that confers resistance to foreign genetic elements such as plasmids and phages and provides a form of acquired immunity. CRISPR spacers recognize and silence these exogenous genetic elements like RNAi in eukaryotic organisms.

CRISPR repeats range in size from 24 to 48 base pairs. They usually show some dyad symmetry, implying the formation of a secondary structure such as a hairpin, but are not truly palindromic. Repeats are separated by spacers of similar length. Some CRISPR spacer sequences exactly match sequences from plasmids and phages, although some spacers match the prokaryote's genome (self-targeting spacers). New spacers can be added rapidly in response to phage infection.

### B. Cas Nucleases

CRISPR-associated (cas) genes are often associated with CRISPR repeat-spacer arrays. As of 2013, more than forty different Cas protein families had been described. Of these protein families, Cas1 appears to be ubiquitous among different CRISPR/Cas systems. Particular combinations of cas genes and repeat structures have been used to define 8 CRISPR subtypes (Ecoli, Ypest, Nmeni, Dvulg, Tneap, Hmari, Apern, and Mtube), some of which are associated with an additional gene module encoding repeat-associated mysterious proteins (RAMPs). More than one CRISPR subtype may occur in a single genome. The sporadic distribution of the CRISPR/Cas subtypes suggests that the system is subject to horizontal gene transfer during microbial evolution.

Exogenous DNA is apparently processed by proteins encoded by Cas genes into small elements (~30 base pairs in length), which are then somehow inserted into the CRISPR locus near the leader sequence. RNAs from the CRISPR loci are constitutively expressed and are processed by Cas proteins to small RNAs composed of individual, exogenously derived sequence elements with a flanking repeat sequence. The RNAs guide other Cas proteins to silence exogenous genetic elements at the RNA or DNA level. Evidence suggests functional diversity among CRISPR subtypes. The Cse (Cas subtype Ecoli) proteins (called CasA-E in E. coli) form a functional complex, Cascade, that processes CRISPR RNA transcripts into spacer-repeat units that Cascade retains. In other prokaryotes, Cas6 processes the CRISPR transcripts. Interestingly, CRISPR-based phage inactivation in E. coli requires Cascade and Cas3, but not Cas1 and Cas2. The Cmr (Cas RAMP module) proteins found in Pyrococcus furiosus and other prokaryotes form a functional complex with small CRISPR RNAs that recognizes and cleaves complementary target RNAs. RNA-guided CRISPR enzymes are classified as type V restriction enzymes.

Cas9 is a nuclease, an enzyme specialized for cutting DNA, with two active cutting sites, one for each strand of the double helix. The team demonstrated that they could disable one or both sites while preserving Cas9's ability to locate its target DNA. One can combine tracrRNA and spacer RNA into a “single-guide RNA” molecule that, mixed with Cas9, can find and cut the correct DNA targets and such synthetic guide RNAs are used for gene editing.

Cas9 proteins are highly enriched in pathogenic and commensal bacteria. CRISPR/Cas-mediated gene regulation may contribute to the regulation of endogenous bacterial genes, particularly during bacterial interaction with eukaryotic hosts. For example, Cas protein Cas9 of Francisella novicida uses a unique, small, CRISPR/Cas-associated RNA (scaRNA) to repress an endogenous transcript encoding a bacterial lipoprotein that is critical for F. novicida to dampen host response and promote virulence. Coinjection of Cas9 mRNA and sgRNAs into the germline (zygotes) can generate mice with mutations. Delivery of Cas9 DNA sequences also is contemplated.

The systems CRISPR/Cas are separated into three classes. Class 1 uses several Cas proteins together with the CRISPR RNAs (crRNA) to build a functional endonuclease. Class 2 CRISPR systems use a single Cas protein with a crRNA. Cpf1 has been recently identified as a Class II, Type V CRISPR/Cas systems containing a 1,300 amino acid protein. See also U.S. Patent Publication 2014/0068797, which is incorporated by reference in its entirety.

In some embodiments, the compositions of the disclosure include a small version of a Cas9 from the bacterium Staphylococcus aureus (UniProt Accession No. J7RUA5). The small version of the Cas9 provides advantages over wild-type or full length Cas9. In some embodiments the Cas9 is a spCas9 (AddGene).

Cas9 Endonuclease Dead, also known as dead Cas9 or dCas9, is a mutant form of Cas9 whose endonuclease activity is removed through point mutations in its endonuclease domains. Similar to its unmutated form, dCas9 is used in CRISPR systems along with gRNAs to target specific genes or nucleotides complementary to the gRNA with PAM sequences that allow Cas9 to bind. Cas9 ordinarily has 2 endonuclease domains called the RuvC and HNH domains. The point mutations D10A and H840A change 2 important residues for endonuclease activity that ultimately results in its deactivation. Although dCas9 lacks endonuclease activity, it is still capable of binding to its guide RNA and the DNA strand that is being targeted because such binding is managed by other domains. This alone is often enough to attenuate if not outright block transcription of the targeted gene if the gRNA positions dCas9 in a way that prevents transcriptional factors and RNA polymerase from accessing the DNA. However, this ability to bind DNA can also be exploited for activation since dCas9 has modifiable regions, typically the N and C terminus of the protein, that can be used to attach transcriptional activators.

C. gRNA

A small guide RNA (sgRNA), or gRNA is an RNA with around 20 nucleotides used to direct Cas9 or dCas9 to their targets. gRNAs contain two major regions of importance for CRISPR systems: the scaffold and spacer regions. The spacer region has nucleotides that are complementary to those found on the target genes, often in the promoter region. The scaffold region is responsible for formation of a complex with (d) Cas9. Together, they bind (d) Cas9 and direct it to the gene(s) of interest. Since the spacer region of a gRNA can be modified for any potential sequence, they give CRISPR systems much more flexibility as any genes and nucleotides with a sequence complementary to the spacer region can become possible targets.

### III. PHARMACEUTICAL FORMULATIONS

The present disclosure provides pharmaceutical compositions. Such compositions comprise a prophylactically or therapeutically effective amount of an agent, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a particular carrier when the pharmaceutical composition is administered intravenously. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulations can include standard carriers such as pharmaceutical grades of mannitol, lactose, starch, magnesium stearate, sodium saccharine, cellulose, magnesium carbonate, etc. Examples of suitable pharmaceutical agents are described in “Remington's Pharmaceutical Sciences.” Such compositions will contain a prophylactically or therapeutically effective amount of the agent, preferably in purified form, together with a suitable amount of carrier so as to provide the form for proper administration to the patient. The formulation should suit the mode of administration, which can be oral, intravenous, intraarterial, intrabuccal, intranasal, nebulized, bronchial inhalation, intra-rectal, vaginal, topical or delivered by mechanical ventilation.

Pharmaceutically acceptable salts include the acid salts and those which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Generally, the ingredients of compositions of the disclosure are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

### IV. EXAMPLES

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Materials and Methods

Cell culture. HEK293T cells (ATCC, CRL-11268) and miniCMV reporter HEK293T cell (Escobar et al., 2022) were cultured in Dulbecco's modified Eagle's medium (DMEM; Gibco, 31-053-028) supplemented with 10% FBS (Sigma, F2442) and 1% penicillin/streptomycin (Gibco, 15140). CD34⁺ human hematopoietic stem cells (HSCs; StemCell, 70060) were cultured in StemSpan Leukemic Cell Culture Kit (StemCell, 09720) and Complete MethoCult Optimum media (StemCell, H4034). All cell lines were maintained at 37° C. with 5% CO₂.

Plasmid construction. For dCas9-encoding vectors, the cloning backbone was modified from pLV-dCas9-p300-P2A-Puro (Addgene, 83889) by inserting an SV40 NLS peptide at the N-terminal of dCas9-p300). NUP98a, NUP98b, and various FG domains were amplified from the pPEP-TEV-cNup98 (amino acids 1-498; Addgene, 38037). HOXA9 (amino acids 164-272) was amplified from pFUW-tetO-HOXA9 (Addgene, 139827) and KDM5A (amino acids 14851690) was partially (amino acids 1485-1622) amplified from pBabe-puro/HA-FLAG-RBP2 (Addgene, 14802) and partially (amino acids 1623-1690) synthesized as a gBlock by IDT. The NUP98 F/S mutant was partly amplified from the IDRFS vector (Ahn et al., 2021) and partially synthesized as a gBlock by IDT. EGFP was amplified from miniCMV-EGFP-hPGK-BSD (Addgene, 188519). The dCas9-NUP98a, dCas9-NUP98b, dCas9-FG, dCas9-F/S. dCas9-NUP98-HOXA9 and dCas9-NUP98-KDM5A plasmids (and associated EGFP harboring variants) were created by subcloning the corresponding amplified fragments into a BamHI-digested plasmid (modified from Addgene, 83889) backbone via NEBuilder HiFi DNA Assembly (NEB, E2621).

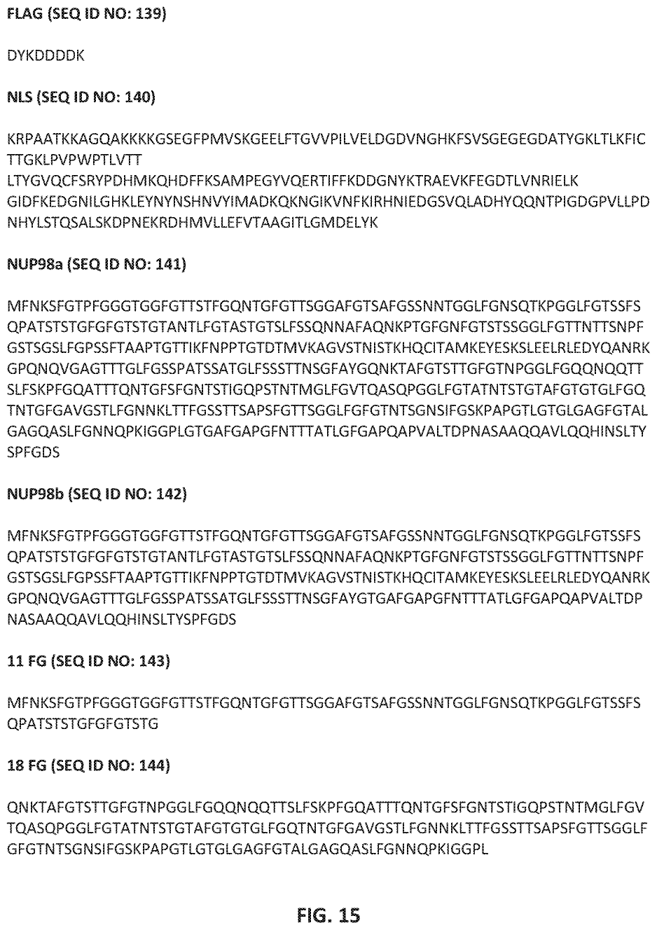

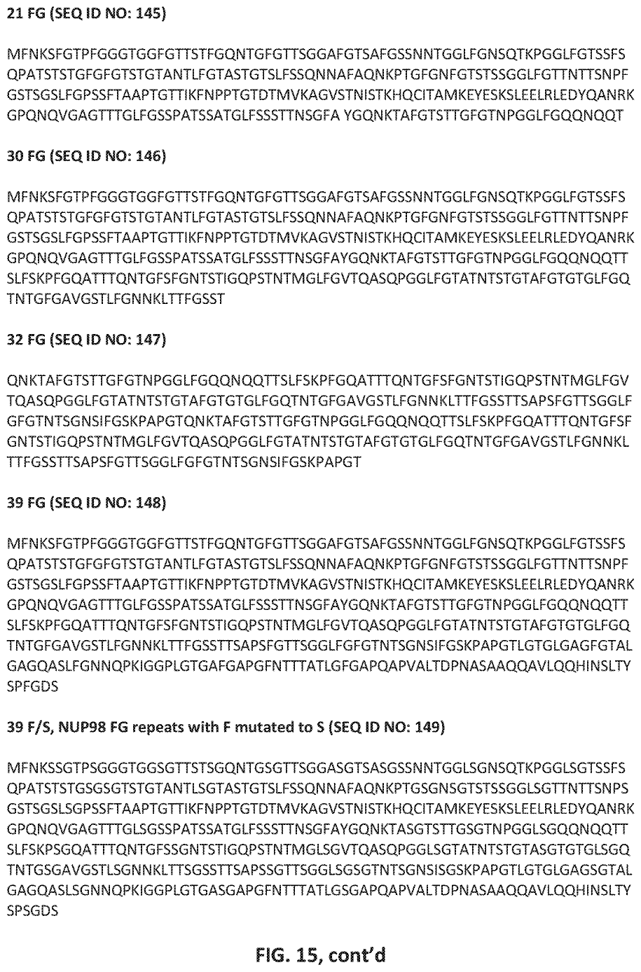

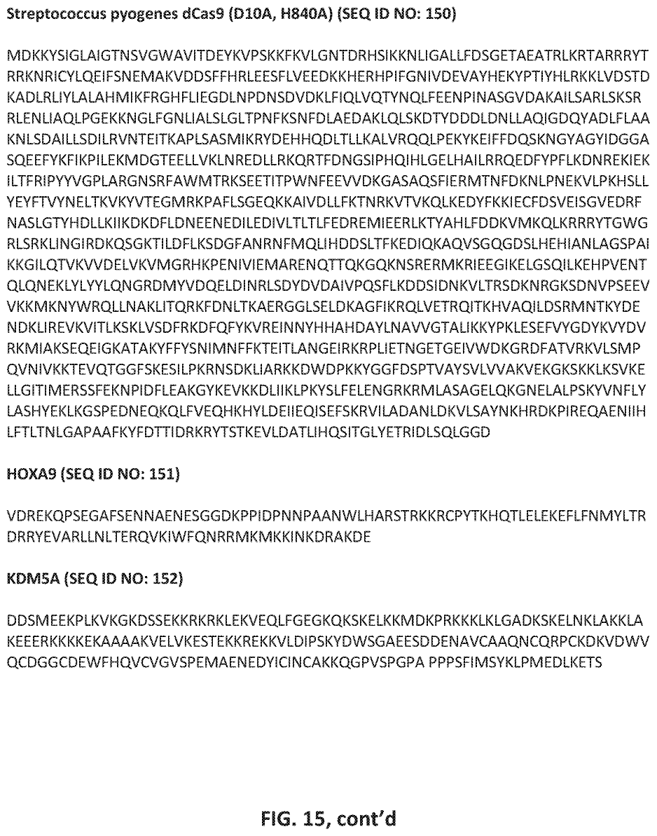

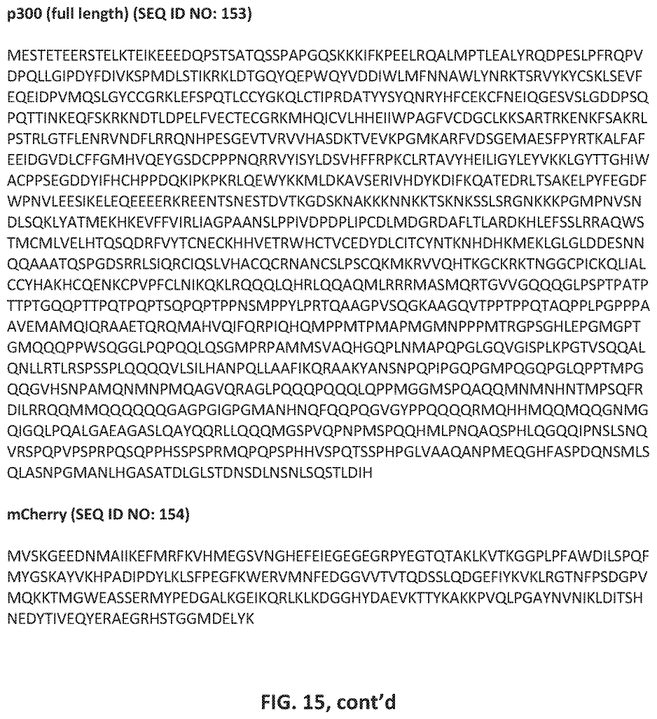

For constructs without dCas9, the cloning backbone was modified from pLV-dCas9-p300-P2A-Puro (Addgene, 83889) by replacing the dCas9-p300-p2a-puro cassette with a hPGK-puro cassette using NEBuilder HiFi DNA Assembly (NEB, E2621). NUP98a-EGFP, NUP98b-EGFP, various FG domains-EGFP, NUP98a/b-HOXA9-EGFP and NUP98a/b-KDM5A-EGFP were subcloned into XbaI/BamHI digested backbone by NEBuilder HiFi DNA Assembly (NEB, E2621). Protein sequences of all dCas9 constructs are shown in FIG. 15. All gRNAs used for non-screening experiments were cloned into the pSPgRNA backbone (Addgene, 47108). All gRNA protospacer targets are listed in Table 1.

TABLE 1

| | | | Genomic | |
|---|---|---|---|---|
| | Protospacer | SEQ | Location | |
| | Sequence | ID | (GRCh38/hg38 | |
| Target Location | (5′-3′) | NO: | Assembly) | Reference |
| IL1RN Promoter A | TGTACTCTCTGAGGTGCTC | 1 | chr2: 113875442- | Perez-Pinera et al., Nat. |
| | | | 113875460 | Methods, 2013 |
| IL1RN Promoter B | ACGCAGATAAGAACCAGTT | 2 | chr2: 113875291- | Perez-Pinera et al., Nat. |
| | | | 113875309 | Methods, 2013 |
| IL1RN Promoter C | CATCAAGTCAGCCATCAGC | 3 | chr2: 113875358- | Perez-Pinera et al., Nat. |
| | | | 113875376 | Methods, 2013 |
| IL1RN Promoter D | GAGTCACCCTCCTGGAAAC | 4 | chr2: 113875326- | Perez-Pinera et al., Nat. |
| | | | 113875344 | Methods, 2013 |
| OCT4 Promoter_A | ACTCCACTGCACTCCAGTCT | 5 | chr6: 31138711- | Hu et al., Nucleic Acids |
| | | | 31138730 | Res., 2014 |
| OCT4 Promoter_B | TCTGTGGGGGACCTGCACTG | 6 | chr6: 31138643- | Hu et al., Nucleic Acids |
| | | | 31138662 | Res., 2014 |
| OCT4 Promoter_C | GGGGCGCCAGTTGTGTCTCC | 7 | chr6: 31138613- | Hu et al., Nucleic Acids |
| | | | 31138632 | Res., 2014 |
| OCT4 Promoter_D | ACACCATTGCCACCACCATT | 8 | chr6: 31138574- | Hu et al., Nucleic Acids |
| | | | 31138593 | Res., 2014 |
| HBG1 promoter_A | GCTAGGGATGAAGAATAAA | 9 | chr11: 5,249,883- | Perez-Pinera et al., Nat |
| | | | 5,249,901 | Methods. 2013 |
| HBG1 promoter_B | TTGACCAATAGCCTTGACA | 10 | chr11: 5,249,958- | Perez-Pinera et al., Nat |
| | | | 5,249,976 | Methods. 2013 |
| HBG1 promoter_C | TGCAAATATCTGTCTGAAA | 11 | chr11: 5,250,020- | Perez-Pinera et al., Nat |
| | | | 5,250,038 | Methods. 2013 |
| HBG1 promoter_D | AAATTAGCAGTATCCTCTT | 12 | chr11: 5,250,066- | Perez-Pinera et al., Nat |
| | | | 5,250,084 | Methods. 2013 |
| OCT4_PE_A | ACTTCAGGTTCAAAGAAGCC | 13 | chr6: 31171948- | Hilton et al, Nat. |
| | | | 31171967 | Biotech. 2015 |
| OCT4_PE_B | CCCTGGGTGGGGAAAACCAG | 14 | chr6: 31171898 | Hilton et al. Nat. |
| | | | 31171917 | Biotech. 2015 |
| OCT4_PE_C | TTTTCCCCACCCAGGGCCTA | 15 | chr6: 31171894 | Hilton et al, Nat. |
| | | | 31171913 | Biotech. 2015 |
| OCT4_PE_D | AGGGAGAACGGGGCCTACCG | 16 | chr6: 31171843 | Hilton et al, Nat. |
| | | | 31171862 | Biotech. 2015 |
| OCT4_PE_E | CAGACATCTAATACCACGGT | 17 | chr6: 31171827 | Hilton et al, Nat. |
| | | | 31171846 | Biotech. 2015 |
| OCT4_PE_F | AGTGATAAGACACCCGCTTT | 18 | chr6: 31171747 | Hilton et al, Nat. |
| | | | 31171766 | Biotech. 2015 |
| OCT4_DE_A | GCATGACAAAGGTGCCGTGA | 19 | chr6: 31173098 | Hilton et al, Nat. |
| | | | 31173117 | Biotech. 2015 |
| OCT4 DE B | GTGCCGTGATGGTTCTGTCC | 20 | chr6: 31173087 | Hilton et al, Nat. |
| | | | 31173106 | Biotech. 2015 |
| OCT4_DE_C | GGAGGAACATGCTTCGGAAC | 21 | chr6: 31173032 | Hilton et al, Nat. |
| | | | 31173051 | Biotech. 2015 |
| OCT4_DE_D | CCTGCCTTTTGGGCAGTTAA | 22 | chr6: 31172987 | Hilton et al, Nat. |
| | | | 31173006 | Biotech. 2015 |
| OCT4_DE_E | TCGGCCTTTAACTGCCCAAA | 23 | chr6: 31172980 | Hilton et al, Nat. |
| | | | 31172999 | Biotech. 2015 |
| OCT4_DE_F | GGTCTGCCGGAAGGTCTACA | 24 | chr6: 31172930 | Hilton et al, Nat. |
| | | | 31172949 | Biotech. 2015 |
| HS2 Enhancer_A | AATATGTCACATTCTGTCTC | 25 | chr11: 5280570- | Hilton et al., Nat. |
| | | | 5280588 | Biotech. 2015 |
| HS2 Enhancer_B | GGACTATGGGAGGTCACTAA | 26 | chr11: 5280878- | Hilton et al., Nat. |
| | | | 5280897 | Biotech. 2015 |
| HS2 Enhancer_C | GAAGGTTACACAGAACCAGA | 27 | chr11: 5280803- | Hilton et al., Nat. |
| | | | 5280822 | Biotech. 2015 |
| HS2 Enhancer_D | GCCCTGTAAGCATCCTGCTG | 28 | chr11: 5280668- | Hilton et al., Nat. |
| | | | 5280687 | Biotech. 2015 |
| MiniCMV | CCGTTCTATGAGATGCTCCG | 29 | N/A | Escobar and Li et al., |
| promoter | | | | ACS synthetic Biology, |
| | | | | 2023 |
| MEIS1_1_A | GCAAGTAGGCTCCTCGGCAG | 30 | chr2: 66435042- | This Study |
| | | | 66435061 | |
| MEIS1_1_B | CACGCACGGGGACTGCAAGC | 31 | chr2: 66435172- | This Study |
| | | | 66435191 | |
| MEIS1_1_C | CCTCTTTGCAAGTGCTGCAC | 32 | chr2: 66435271- | This Study |
| | | | 66435290 | |
| MEIS1_1_D | ATTCAAGGGAAGCGAGCGCT | 33 | chr2: 66435354- | This Study |
| | | | 66435373 | |
| MEIS1_1_E | CACAGGCGAGGTTGTCAACG | 34 | chr2: 66435453- | This Study |
| | | | 66435472 | |
| MEIS1_2_A | TTATTACTACTTGCCCTCAG | 35 | chr2: 66464535- | This Study |
| | | | 66464554 | |
| MEIS1_2_B | TCAAGCTAGTCTTCAGCAAA | 36 | chr2: 66464880- | This Study |
| | | | 66464899 | |
| MEIS1_2_C | TTAAACTTTAATTGCATTAA | 37 | chr2: 66464970- | This Study |
| | | | 66464989 | |
| MEIS1_2_D | GGAGTGCTGTTAGACAACAA | 38 | chr2: 66465095- | This Study |
| | | | 66465114 | |
| MEIS1_3_A | TAAATCGTTACTCAGTCATC | 39 | chr2: 66469490- | This Study |
| | | | 66469509 | |
| MEIS1_3_B | CCACCTATCCCATTATTCAT | 40 | chr2: 66469589- | This Study |
| | | | 66469608 | |
| MEIS1_3_C | ACCACTCTCAAGTTGTCCAT | 41 | chr2: 66469776- | This Study |
| | | | 66469795 | |
| MEIS1_3_D | GGAAAGGTGAGTATATTACA | 42 | chr2: 66470462- | This Study |
| | | | 66470481 | |
| MEIS1_3_E | TATATTTAGGGGCACGAACT | 43 | chr2: 66470599- | This Study |
| | | | 66470618 | |
| MEIS1_4_A | ACACGGACTTCATGACACAT | 44 | chr2: 66508648- | This Study |
| | | | 66508667 | |
| MEIS1_4_B | TGTCAGAAACCATTAGCGGT | 45 | chr2: 66508744- | This Study |
| | | | 66508763 | |
| MEIS1_4_C | AGCCTCATTAGGATAGCAGT | 46 | chr2: 66508834- | This Study |
| | | | 66508853 | |
| MEIS1_4_D | CTGCTGACATTTTACAAATG | 47 | chr2: 66508955- | This Study |
| | | | 66508974 | |
| MEIS1_5_A | TTACAGTGTAAAAACGAATT | 48 | chr2: 66523078- | This Study |
| | | | 66523097 | |
| MEIS1_5_B | AGTTGCCGTTTATGTACTGA | 49 | chr2: 66523172- | This Study |
| | | | 66523191 | |
| MEIS1_5_C | AGGCAAAACCAAGACTATGC | 59 | chr2: 66523248- | This Study |
| | | | 66523267 | |
| MEIS1_5_D | GCTTCTGCATGCAAGGTTGT | 51 | chr2: 66523337- | This Study |
| | | | 66523356 | |
| MEIS1_6_A | AATTTTAGCTTTACAAAGGG | 52 | chr2: 66536916- | This Study |
| | | | 66536935 | |
| MEIS1_6_B | TTGACCTTTAATGGTAAGCT | 53 | chr2: 66537011- | This Study |
| | | | 66537030 | |
| MEIS1_6_C | GCTTGAGAGTGTCTGTTAGC | 54 | chr2: 66537080- | This Study |
| | | | 66537099 | |
| MEIS1_6_D | TGAATGTTCTTTACTCAAAT | 55 | chr2: 66537196- | This Study |
| | | | 66537215 | |
| MEIS1_6_E | GATGAGCCCAGGCTAATTCA | 56 | chr2: 66537274- | This Study |
| | | | 66537293 | |
| MEIS1_7_A | GTTTTATCAGCACAACTCCT | 57 | chr2: 66564675- | This Study |
| | | | 66564694 | |
| MEIS1_7_B | ACAACAATTACAGGGGGAGG | 58 | chr2: 66564871- | This Study |
| | | | 66564890 | |
| MEIS1_7_C | GGCCACCCCATCTATAAACA | 59 | chr2: 66564982- | This Study |
| | | | 66565001 | |
| MEIS1_7_D | GAGGTTACCTATGTACTTTT | 60 | chr2: 66565039- | This Study |
| | | | 66565058 | |
| MEIS1_8_A | GAAATGTCACTAATTAGGAG | 61 | chr2: 66567056- | This Study |
| | | | 66567075 | |
| MEIS1_8_B | CCAGTGACCTTTGAGTACAC | 62 | chr2: 66567260- | This Study |
| | | | 66567279 | |
| MEIS1_8_C | CGGTTGGACTGGTCTATCAT | 63 | chr2: 66567487- | This Study |
| | | | 66567506 | |
| MEIS1_8_D | GTGTTGTTATGAAACGAAAC | 64 | chr2: 66567645- | This Study |
| | | | 66567664 | |
| MEIS1_8_E | TATGACCACTCCCTGGTGCA | 65 | chr2: 66567831- | This Study |
| | | | 66567850 | |
| MLLT3_1_A | AGTCCTTAGCTAGTACGCAC | 66 | chr9: 20622873- | This Study |
| | | | 20622892 | |
| MLLT3_1_B | AGGGCGAGCAGAGTTTACAC | 67 | chr9: 20622773- | This Study |
| | | | 20622792 | |
| MLLT3_1_C | GGAACCTGTTACTTTAAGCG | 68 | chr9: 20622682- | This Study |
| | | | 20622701 | |
| MLLT3_1_D | AGGCAGAGCGAGCGAGCCAG | 69 | chr9: 20622588- | This Study |
| | | | 20622607 | |
| MLLT3_1_E | CGCAATCATCTTCTTTACCC | 70 | chr9: 20622483- | This Study |
| | | | 20622502 | |
| MLLT3_2_A | CTGTCCATTCACCATAAGAA | 71 | chr9: 20609813- | This Study |
| | | | 20609832 | |
| MLLT3_2_B | GTGTAACTTAATCCCTTATT | 72 | chr9: 20609730- | This Study |
| | | | 20609749 | |
| MLLT3_2_C | ATCAACTATTACTTGTAGAT | 73 | chr9: 20609664- | This Study |
| | | | 20609683 | |
| MLLT3_2_D | ATGGTCAAGAGCCATAATTG | 74 | chr9: 20609589- | This Study |
| | | | 20609608 | |
| MLLT3_2_E | AATTAAGTGACACAATTTCA | 75 | chr9: 20609514- | This Study |
| | | | 20609533 | |
| MLLT3_3_A | GCTGTTTTAAAAGTACGGGT | 76 | chr9: 20606425- | This Study |
| | | | 20606444 | |
| MLLT3_3_B | AAGGCGGATTCATGAGTGGA | 77 | chr9: 20606336- | This Study |
| | | | 20606355 | |
| MLLT3_3_C | AAGAAGCTGGTACATAGGAC | 78 | chr9: 20606254- | This Study |
| | | | 20606273 | |
| MLLT3_3_D | ACTGCCTGTAAAATCTCCCA | 79 | chr9: 20606186- | This Study |
| | | | 20606205 | |
| MLLT3_3_E | CCAGCCATAGAGAATACGCA | 80 | chr9: 20606115- | This Study |
| | | | 20606134 | |
| MLLT3_4_A | TCTCAGAAGCATATTGGCAG | 81 | chr9: 20552124- | This Study |
| | | | 20552143 | |
| MLLT3_4_B | TATTTGTTTGTGCACGTAGG | 82 | chr9: 20551925- | This Study |
| | | | 20551944 | |
| MLLT3_4_C | GATACAAACCATTTACCTGG | 83 | chr9: 20551741- | This Study |
| | | | 20551760 | |
| MLLT3_4_D | TCAGCTTTGAAGGTTGCCTT | 84 | chr9: 20551586- | This Study |
| | | | 20551605 | |
| MLLT3_4_E | TTACATGGACCTGCTTTCCC | 85 | chr9: 20551431- | This Study |
| | | | 20551450 | |
| MLLT3_5_A | GAGTCATAAGCTCCAGTGCA | 86 | chr9: 20537473- | This Study |
| | | | 20537492 | |
| MLLT3_5_B | GTACTCAATGTTATTAAAGC | 87 | chr9: 20537203- | This Study |
| | | | 20537222 | |
| MLLT3_5_C | GCACCTAGGTTTCATGGTCC | 88 | chr9: 20537015- | This Study |
| | | | 20537034 | |
| MLLT3_5_D | GGAGTCGTCCCATACTGGAG | 89 | chr9: 20536807- | This Study |
| | | | 20536826 | |
| MLLT3_5_E | ACTACAAAAGATTACCCTTC | 90 | chr9: 20536690- | This Study |
| | | | 20536709 | |
| MLLT3_6_A | GATGGACCCTTCTAAAATAG | 91 | chr9: 20487593- | This Study |
| | | | 20487612 | |
| MLLT3_6_B | ACGTTTGACATGGGAAAGCA | 92 | chr9: 20487499- | This Study |
| | | | 20487518 | |
| MLLT3_6_C | ATCATAGGACTTTTGCTACT | 93 | chr9: 20487339- | This Study |
| | | | 20487358 | |
| MLLT3_6_D | CTCCTTACATTTAGCAGACC | 94 | chr9: 20487225- | This Study |
| | | | 20487244 | |
| MLLT3_6_E | TCTGCTTCGCAACTTTTATT | 95 | chr9: 20487155- | This Study |
| | | | 20487174 | |
| MLLT3_7_A | TGACTTTAAGATTCAACTAC | 96 | chr9: 20469594- | This Study |
| | | | 20469613 | |
| MLLT3_7_B | AAATCAACTGTTTGCACATT | 97 | chr9: 20469322- | This Study |
| | | | 20469341 | |
| MLLT3_7_C | AGGTGACTCCCAAGTAAGAA | 98 | chr9: 20469117- | This Study |
| | | | 20469136 | |
| MLLT3_7_D | CATCTGCTGTTAACACGATC | 99 | chr9: 20468862- | This Study |
| | | | 20468881 | |
| MLLT3_7_E | GCTGGCTTCTTATGTGACAG | 100 | chr9: 20468697- | This Study |
| | | | 20468716 | |
| MLLT3_8_A | AGGAAGAACTGTAACCGTTC | 101 | chr9: 20408505- | This Study |
| | | | 20408524 | |
| MLLT3_8_B | TGTTGGGTGTGATCACACTG | 102 | chr9: 20408368- | This Study |
| | | | 20408387 | |
| MLLT3_8_C | CCGTGTTTCAGTACATACTA | 103 | chr9: 20408231- | This Study |
| | | | 20408250 | |
| MLLT3_8_D | AGCTGGTTGTCATCAACAAT | 104 | chr9: 20408115- | This Study |
| | | | 20408134 | |
| MLLT3_8_E | CCAACAATGGGACTCAACAT | 105 | chr9: 20407923- | This Study |
| | | | 20407942 | |
| MLLT3_9_A | TCAAAAGCGAGCACCTGTCA | 106 | chr9: 20373430- | This Study |
| | | | 20373449 | |
| MLLT3_9_B | ACCAAAACAATGGGCCTACC | 107 | chr9: 20373284- | This Study |
| | | | 20373303 | |
| MLLT3_9_C | ATTAGTTTGCTCATAGAGTG | 108 | chr9: 20373098- | This Study |
| | | | 20373117 | |
| MLLT3_9_D | AAAGTTATATTTATGCTATA | 109 | chr9: 20372910- | This Study |
| | | | 20372929 | |
| MLLT3_9_E | GCTGATGACTACTTATCGAT | 110 | chr9: 20372762- | This Study |
| | | | 20372781 | |

Live cell imaging. 4e5 HEK293T cells were seeded in 35 mm glass-bottom petri dishes with 10 mm microwells (MatTek, P35G-1.5-10-C). In experiments with only one fluorophore and no dCas9/gRNA, 500 ng of indicated EGFP/mCherry containing vectors were used. In experiments using dCas9 and gRNAs, 375 ng of dCas9 and EGFP fusion encoding vector and 125 ng of gRNA vector were used. In experiments using two fluorophores (i.e., p300-mCherry and FG-EGFP), 250 ng of EGFP containing vector and 250 ng of mCherry containing vector were used. Cells were transiently transfected using Lipofectamine 3000 (Invitrogen, L3000015). 20 hours post-transfection cells were stained using NucBlue Live ReadyProbes Reagent (Hoechst 33342; Invitrogen, R37605) for 20 minutes. After incubation cells were imaged using a Nikon ECLIPSE Ti2-E (541094). Fluorescence quantification was performed using NIS-Elements AR (v5.10.01).

Reverse-transcription quantitative PCR (RT-qPCR). HEK293T cells were transiently transfected with respective dCas9 expression vector (375 ng) and single gRNA vectors or equimolar pooled gRNA expression vectors (125 ng) in 24-well plates using Lipofectamine 3000 (Invitrogen, L3000015). RNA was extracted from HEK293T cells ~72 hours post-transfection using the Qiagen RNeasy Plus mini kit (Qiagen, 74136). 1 μg of RNA was used for cDNA synthesis using the iScript advanced cDNA synthesis kit (Bio-Rad, 1725038). RT-qPCR was performed using Luna Universal qPCR Master Mix (NEB, M3003E) on a CFX96 Real-Time PCR Detection System with a C1000 Thermal Cycler (Bio-Rad, 1855195). Baselines were subtracted using the baseline subtraction curve fit analysis mode and thresholds were automatically calculated using the Bio-Rad CFX Manager software (version 2.1). Results are expressed as fold change above mock-transfected cells after normalization to GAPDH expression using the ΔΔCt method. All qPCR primers and conditions are listed in Table 2.

TABLE 2
Quantitative reverse transcription PCR and CUT&RUN-qPCR primers and conditions

| | | SEQ | | SEQ | | |
|---|---|---|---|---|---|---|
| Target | Forward Primer (5′-3′) | ID NO: | Reverse Primer (5′-3′) | ID NO: | Cycling Parameters | |
| GAPDH | CAATGACCCCTTCATTGACC | 111 | TTGATTTTGGAGGGATCTCG | 120 | 95 °C 30 sec | 40 x |
| | | | | | 95 °C 10 sec | |
| | | | | | 60 °C 30 sec | |
| OCT4 | CGAAAGAGAAAGCGAACCAGTATC | 112 | CGTTGTGCATAGTCGCTGCTTGA | 121 | 95 °C 30 sec | 40 x |
| | GAGAAC | | TCGC | | 95 °C 10 sec | |
| | | | | | 60 °C 30 sec | |
| IL1RN | GGAATCCATGGAGGGAAGAT | 113 | TGTTCTCGCTCAGGTCAGTG | 122 | 95 °C 30 sec | 40 x |
| | | | | | 95 °C 10 sec | |
| | | | | | 60 °C 30 sec | |
| HBG1 | GCTGAGTGAACTGCACTGTGA | 114 | GAATTCTTTGCCGAAATGGA | 123 | 95 °C 30 sec | 40 x |
| | | | | | 95 °C 10 sec | |
| | | | | | 60 °C 30 sec | |
| OCT4 | GTCTGGGCAACAAAGTGAGA | 115 | CAATGGTGTCTGTGGAAGGG | 124 | 95 °C 30 sec | 40 x |
| CUT & | | | | | 95 °C 10 sec | |
| RUN-qPCR | | | | | 60 °C 10 sec | |
| IL1RN | GGCATCAAGTCAGCCATCA | 116 | AGAGCACCTCAGAGAGTACAA | 125 | 95 °C 30 sec | 40 x |
| CUT & | | | | | 95 °C 10 sec | |
| RUN-qPCR | | | | | 60 °C 30 sec | |
| HBG1 | CTTCAGCAGTTCCACACACT | 117 | GTCCTTCCTTCCCTCCCT | 126 | 95 °C 30 sec | 40 x |
| CUT & | | | | | 95 °C 10 sec | |
| RUN-qPCR | | | | | 60 °C 30 sec | |
| MLLT3 3C | TGTTAGAGCATTCTTTGAGGA | 118 | CTATTTTGTGTGCTACCTAT | 127 | 95 °C 30 sec | 45 x |
| qPCR | | | CAATTTTCCATTAATTGAATC | 128 | 95 °C 10 sec | |
| | | | ATATAAGCTATCAAACAGATC | 129 | 60 °C 30 sec | |
| | | | ATTTATGTCAAAAATCAAGGC | 130 | | |
| | | | CTGTATAACTGAGGTGAAAGC | 131 | | |
| | | | TTCTTCCTATCCCTGTACTCC | 132 | | |
| | | | TTGTGGGGGAGACACCTTAAA | 133 | | |
| MEIS1 3C | TTCTCCCTCCTTTTCCTCCAC | 119 | TAAAGTATGTACCTACTGTGG | 134 | 95 °C 30 sec | 45 x |
| qPCR | | | CTCAAACCCCTTCAGATTAAT | 135 | 95 °C 10 sec | |
| | | | CCACACCAGGCTAAGTTTTGT | 136 | 60 °C 30 sec | |
| | | | AATGGTACATTTGGTGAGGAG | 137 | | |
| | | | AGGCTCACTAAGGGTGATTAC | 138 | | |

MiniCMV reporter assay. The miniCMV HEK293T reporter cell line (Escobar et al., 2022) was seeded into 24 well plates and transfected at 60-70% confluency the next day using Lipofectamine 3000 (ThermoFisher, L3000015) using 375 ng of indicated Cas plasmids and 125 ng associated gRNAs as per manufacturer's protocol. EGFP intensity was analyzed ~72 hours post-transfection using a Sony SA3800 flow cytometer and compared to a non-targeting gRNA control.

Lentivirus production. HEK293T cells were seeded at ~40% confluency in 10-cm plates one day prior to transfection. Cells were transfected with lentiviral production plasmids at ~80-90% confluency on the next day. For each transfection, 10 μg of plasmid containing the vector of interest, 10 μg of pMD2.G (Addgene, 12259), and 15 μg of psPAX2 (Addgene, 12260) were transfected using Lipofectamine 3000 (Invitrogen, L3000015). Six hours post-transfection the media was changed. The supernatant was harvested at ~24 and ~48 hours post-transfection and filtered through a 0.45-μm PVDF filter (Millipore, SLGVM33RS). Viruses were concentrated using Lenti-X Concentrator (Takara, 631232), aliquoted, and stored at −80° C. Lentiviral titers were measured by the Lenti-X qRT-PCR Titration Kit (Takara, 631232).

Lentiviral transduction. 24-well plates were treated with 20 μg/ml RetroNectin (Takara Bio, T100B) as per manufacturer's instruction. 2e6 CD34⁺ human HSCs (StemCell, 70060) were then mixed with 2e9 lentiviral particles and Lentiboost-P (1:100 dilution, Siron Biotech), and were then seeded onto the RetroNectin-treated plates. After centrifugation at 800 g at 4° C. for 90 mins, plates were placed at 37° C. overnight. The cell medium was changed and cells were cultured for another ~48 hours. The transduced cells were imaged using a fluorescent microscope (Nikon ECLIPSE Ti2-E; 541094) and EGFP-positive cells were isolated using FACS cell sorting (Sony Biotechnology, MA900) and cultured in Complete MethoCult Optimum media (StemCell, H4034) for 2 weeks. HEK293T cells were transduced with lentiviruses encoding EGFP, NUP98a-HOXA9 or NUP98a-KDM5A in 6-well plates at a MOI of 10. Briefly, 1e6 cells in 2 ml of media supplemented with 8 μg per ml polybrene (Sigma, TR-1003-G) were added to each well. Then, 48 hours post-transduction, cells were passaged and EGFP positive cells were isolated by FACS cell sorting (Sony Biotechnology, MA900) and cultured in complete DMEM media for 2 weeks with passaging every 3 days.

CUT & RUN assays. CUT&RUN assays were carried out using the Epicypher CUTANA ChIP/CUT&RUN Kit (Epicypher 14-1048). Briefly, 5.0e5 transfected cells were harvested, immobilized on concanavalin A beads, and permeabilized in 0.01% digitonin cell permeabilization buffer. The cell-bead conjugate mixture was then divided equally into 2 aliquots and incubated in 50 μl antibody buffer with either 0.5 μg of anti-H3K27ac antibody (Abcam, 4729) or 0.5 μg of control rabbit IgG antibody (Abcam, 37415) overnight at 4° C. After washing the beads, pAG-MNase was added to the immobilized cells and the solution was incubated for 2 hours at 4° C. to digest and release chromatin DNA. For CUT&RUN-qPCR assays, 1 μl of purified DNA from both H3K27ac antibody treatment and rabbit IgG control treatment was then assayed using qPCR. Relative enrichment of H3K27ac is expressed as fold change relative to the corresponding rabbit IgG control samples using the ΔΔCt method. All qPCR primers and conditions are listed in Table 2.

Mass spectrometry. HEK293T cells were co-transfected with the indicated dCas9 or dCas9-fusion expression vectors (15 μg) and gRNA constructs (5 μg) in 15-cm plates using Lipofectamine 3000 (Invitrogen, L3000015) as per the manufacturer's instruction. ~48 hours post-transfection, cells were crosslinked using a 0.5% final concentration of formaldehyde for 8 min at 37° C. and quenched with glycine added to a final concentration of 200 mM. Cells were then washed twice with 1×PBS and suspended in 2.5 ml NETN buffer (50 mM Tris pH 7.3, 170 mM NaCl, 1 mM EDTA, 0.5% NP-40). Cells were sonicated using Bioruptor Pico (Diagenode, B01060010) with six cycles of 30 s burst and 59 s off, followed by centrifugation at 20,000 g at 4° C. for 20 minutes. The supernatant containing whole cell extract was collected and centrifuged again at 20,000 g at 4° C. for 20 minutes. The recollected supernatant was mixed with 5 μg of anti-FLAG (Sigma-Aldrich, F1804) antibody and incubated at 4° C. for 1 hour with gentle shaking. After a centrifuge of 20, 000 g at 4° C. for 20 minutes, the supernatant was collected. FLAG-tagged proteins were immunoprecipitated via incubation of 20 μl of ProteinA/G Magnetic Beads (ThermoFisher Scientific, 88802) at 4° C. for 1 hour. The beads were washed with 1 ml NETN buffer twice and centrifuged at 1,000 g for 1 min. The supernatant was removed completely.

The immunoprecipitated samples were resolved on NuPAGE 10% Bis-Tris Gel (Life Technologies), each lane was excised into 6 equal pieces and combined into two peptide pools after in-gel digestion using trypsin enzyme. The peptides were dried in a speed vac and dissolved in 5% methanol containing 0.1% formic acid buffer. The LC-MS/MS analysis was carried out using the nano-LC 1000 system coupled to Orbitrap Fusion mass spectrometer (Thermo Scientific). Peptides were eluted on an analytical column (20 cm×75 μm I.D.) filled with Reprosil-Pur Basic C18 (1.9 μm, Dr. Maisch GmbH, Germany) using 110 minutes discontinuous gradient of 90% acetonitrile buffer (B) in 0.1% formic acid at 200 nl/min (2-30% B: 86 min, 30-60% B: 6 min, 60-90% B: 8 min, 90-50% B: 10 min). The full MS scan was performed in Orbitrap analyzer in the range of 300-1400 m/z at 120,000 resolution followed by an IonTrap HCD-MS2 fragmentation for a cycle time of 3 seconds with precursor isolation window of 3 m/z, collision energy 30%, AGC of 50,000, maximum injection time of 30 ms.

The MS raw data were searched using Proteome Discoverer 2.0 software (Thermo Scientific) with Mascot algorithm against human NCBI RefSeq database updated 2020_0324 containing additional sequences of -dCas9, dCas9-NUP98a and dCas9-NUP98b. The precursor ion tolerance and product ion tolerance were set to 20 ppm and 0.5 Da, respectively. Maximum cleavage of 2 with Trypsin enzyme, dynamic modification of oxidation (M), protein N-term acetylation, deamidation (N/Q) and destreak (C) was allowed. The peptides identified from the mascot result file were validated with a 5% false discovery rate (FDR). The gene product inference and quantification were done with label-free iBAQ approach using the ‘gpGrouper’ algorithm. For statistical assessment, missing value imputation was employed through sampling a normal distribution N (μ-1.8 σ, 0.8σ), where μ, σ are the mean and standard deviation of the quantified values. For differential analysis, the inventors used the moderated t-test and log 2 fold changes as implemented in the R package limma (Ritchie et al., 2015) and multiple-hypothesis testing correction was performed with the Benjamini-Hochberg procedure.

ChIP-sequencing and analysis. HEK293T cells were transfected with indicated NUP98 fusions (10 μg) or cotransfected with indicated dCas9 fusion (7.5 μg) expression vectors and gRNA (2.5 μg) constructs in 10 cm plates in biological duplicates for each condition tested. ~72 hours post-transfection, cells were cross-linked for 10 min at RT using 1% formaldehyde (Sigma F8775-25ML) and then the reaction was stopped by the addition of glycine to a final concentration of 125 mM. Cells were harvested and washed with ice cold 1×PBS and suspended in Farnham lysis buffer (5 mM PIPES pH 8.0, 85 mM KCl, 0.5% NP-40) supplemented with protease inhibitor (Thermo Scientific, A32965). Cells were then pelleted and resuspended in RIPA buffer (1×PBS, 1% NP-40, 0.5% sodium deoxycholate, 0.1% SDS) supplemented with protease inhibitor. Approximately 2.5e7 cells were used for each ChIP experiment. Chromatin in RIPA buffer was sheared to a median fragment size of around 250 bp using a Bioruptor XL (Diagenode). Two μg of α-FLAG antibody (Sigma-Aldrich, F1804) was incubated with 50 μl Mouse IgG magnetic beads (Life Technologies, 11202D) for ~16 hours at 4° C., respectively. Antibody-linked magnetic beads were washed 3 times with PBS/BSA buffer (1×PBS and 5 mg/ml BSA) and sheared chromatin was incubated with corresponding antibody-linked magnetic beads at 4° C. overnight and then washed 5 times with LiCl IP wash buffer (100 mM Tris pH 7.5, 500 mM LiCl, 1% NP-40, 1% sodium deoxycholate). Cross-links were then reversed via overnight incubation at 65° C. and DNA was purified using QIAquick PCR purification kit (Qiagen, 28106) for ChIP-seq. Input DNA was prepared from ~1.0e6 cells.

ChIP DNA samples were quantified using Qubit 2.0 Fluorometer (ThermoFisher Scientific) and the DNA integrity was checked with 4200 TapeStation (Agilent Technologies). ChIP-seq libraries were prepared using the NEB NextUltra DNA Library Preparation kit (NEB, 7645). Briefly, the ChIP DNA was end-repaired and adapters were ligated after adenylation of the 3′ ends. Adapter-ligated DNA was size-selected, followed by clean up, and limited cycle PCR enrichment. The ChIP library was validated using Agilent TapeStation (Agilent) and quantified using Qubit 2.0 Fluorometer as well as real time PCR (KAPA Biosystems). The sequencing libraries were multiplexed and clustered onto one lane of a flowcell. After clustering, the flowcell was loaded on the Illumina HiSeq 4000 equivalent instrument. Sequencing was performed using a 2×150 Paired End (PE) configuration. Image analysis and base calling were conducted by the HiSeq Control Software. Raw sequence data (.bcl files) generated from Illumina HiSeq was converted into Fastq files and de-multiplexed using the bcl2fastq (v2.17). One mismatch was allowed for index sequence identification. For ChIP-seq analysis, reads were aligned to the GRCh38 reference genome using Bowtie2 (v2.4.5) (Langmead & Salzberg, 2012) and peaks were called using MACS2 (v2.2.7.1) (Zhang et al., 2008) with a q-value≤0.01. Resulting peaks were assessed and analyzed with deepTools (v3.5.4) (Ramirez et al., 2016), DiffBind (v3.8.4) (Ross-Innes et al., 2012) and ChIPSeeker (v1.34.1) (Wang et al., 2022).

3C-qPCR. 1.0e7 cells were harvested 72 h post-transfection, crosslinked in 9.5 ml of 1% formaldehyde in PBS with 10% FBS (Sigma-Aldrich, F8775) and then incubated at room temperature for 10 min. 1.425 ml of ice-cold 1M glycine was added to a final concentration of 130 mM glycine to quench crosslinking similar to previous methods (Hagege et al., 2007). Briefly, crosslinked cells were then pelleted and then lysed in 5 ml of lysis buffer (10 mM Tris-HCl, pH 7.5; 10 mM NaCl; 5 mM MgCl2; 0.1 mM EGTA; 1× complete protease inhibitor; Fisher, A32965) on ice for 10 min, and then centrifuged for 5 min at 400 g at 4° C. for nuclei extraction. The extracted nuclei were transferred to 1× CutSmart digestion buffer (NEB, B7204) with 0.3% SDS (Invitrogen 24730020) and then shaken at 900 RPM for 1 h at 37° C., after which, 2% Triton X-100 (Sigma-Aldrich, T9284) was added, and another 1 h incubation with shaking at 900 RPM at 37° C. was performed. 400 U of HindIII (NEB R0104) was then added, and the nuclear DNA was digested at 37° C. while shaking at 900 RPM overnight. The solution was then brought to 7 ml total volume in T4 ligation buffer (NEB B0202) with a final concentration of 1.6% SDS and 1% Triton X-100. The mixture was then gently shaken at 37° C. for 1 h. 100 Weiss units of T4 DNA ligase (NEB M0202) were then added to the nuclear DNA, and the solution was ligated for 4 h at 16° C. and then for 30 min at room temperature. The reaction was then incubated with 300 μg of proteinase K (Qiagen 19131) at 65° C. overnight. The next day 300 μg of RNase (Qiagen 9101) was added and the solution was incubated at 37° C. for 45 min. The nuclear DNA was then purified using phenol-chloroform (Fisher BP17521400) extraction. 1000 ng of extracted DNA was used for qPCR. Results are expressed as fold change above cells transfected with the dCas9 control plasmid after normalization to GAPDH expression using the ΔΔCt method. All qPCR primers and conditions are listed in Table 2.

RNA sequencing and analysis. RNA sequencing (RNA-seq) was performed in duplicate for each experimental condition. RNA was isolated from transduced CD34⁺ human hematopoietic stem cells using the RNeasy Plus mini kit (Qiagen, 74136). Total RNA samples were normalized to 10 ng each, based on picogreen quantitation. RNA-seq libraries were constructed using the Takara SMARTer Stranded Total RNA-Seq Kit v3-Pico Input Mammalian (Takara, 634485). The Switching Mechanism at 5′ end of RNA template (SMARTer) cDNA synthesis technology generates directionally identifiable ds-cDNA, incorporated with Illumina sequencing adapters and sample barcodes. Following cDNA synthesis and purification, ribosomal cDNA reduction is achieved by using probes (R-Probes v3) which target mammalian ribosomal cDNA and human mitochondrial ribosomal cDNA and ZapR v3 cleaves these fragments. Following amplification of the library using universal primers and a final purification using Takara NucleoMag NGS Clean-up and Size Select beads, the final library is ready for quantification and sequencing. Read 1 sequenced reads map to the antisense strand of the original RNA. The resulting libraries were quantitated by picogreen and fragment size assessed with the Agilent 2100 Bioanalyzer.

All samples were pooled equimolarly and re-quantitated by qPCR using the Applied Biosystems ViiA7 Quantitative PCR instrument and a KAPA Library Quant Kit (p/n KK4824), and also re-assessed on the Bioanalyzer. Cluster Generation by Exclusion Amplification (ExAMP): Using the concentration from the ViiA7 TM qPCR machine above, 150 μM of equimolarly pooled library is loaded onto one lane of the NovaSeq S1 flowcell (Illumina p/n 20028317) following the XP Workflow protocol (Illumina kit p/n 20021664) and amplified by exclusion amplification onto a nanowell-designed, patterned flowcell using the Illumina NovaSeq 6000 sequencing instrument. A combination of PhiX Control v3 adapter-ligated library (Illumina p/n FC-110-3001) and RNA library are spiked-in at 10% by weight to ensure balanced diversity and to monitor clustering and sequencing performance. A paired-end 150 bp cycle run was used to sequence the flowcell on a NovaSeq 6000 Sequencing System. An average of 60 million read pairs per sample was sequenced. Fastq file generation was executed using Illumina's cloud-based informatics platform, BaseSpace Sequencing Hub. Reads were aligned to the GRCh38 transcriptome using HISAT2 (2.2.1) (Kim et al., 2019). Transcript abundance was calculated using feature Counts from the subread package (v2.0.3) (Liao et al., 2014), and differential expression was determined in R studio (v2022.07.2, R 4.2.2) using the DESeq2 (v1.38.3) (Love et al., 2014) analysis package with default parameters. KEGG analysis was performed using clusterProfiler (v4.6.0) (Wu et al., 2021) and sequence read coverage was visualized by the IGV (2.16.0) (Robinson et al., 2023).

### Example 2—Results

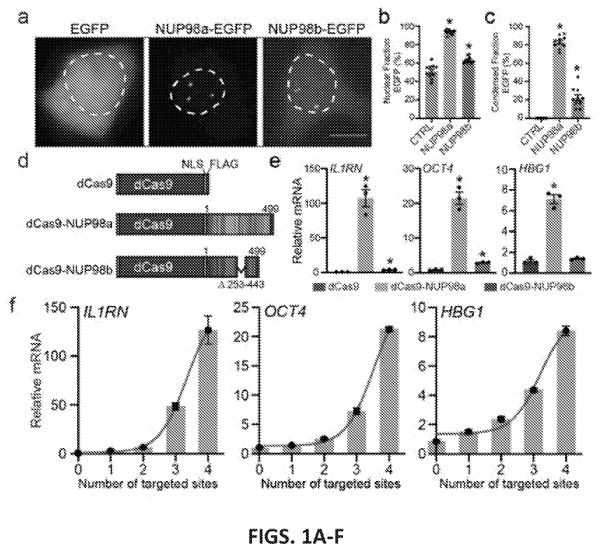

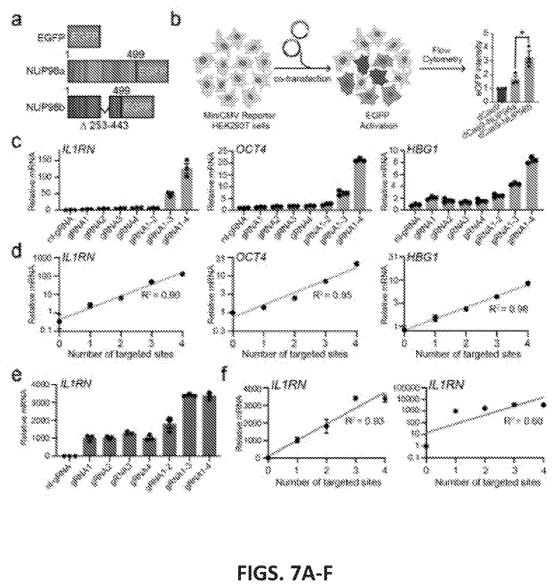

NUP98 FG repeats cooperatively activate human promoters. NUP98 fusions lead to mislocalization of the N-terminal NUP98 FG repeat domain and global transcriptional dysregulation in human and murine cells (Chandra et al., 2022; Terlecki-Zaniewicz et al., 2021). To functionally clarify the aberrant behavior of NUP98 FG repeats, the inventors first fused EGFP to the C-terminus of two different NUP98 FG repeat compositions that have been observed in human leukemias (Kasper et al., 1999) (NUP98a, 39 FG repeats and NUP98b, 21 FG repeats; FIG. 7a) and then monitored how these fusions behaved when transfected into human HEK293T cells. Virtually all (~98%) NUP98a-EGFP transfected cells exhibited sharp, clustered EGFP puncta in nuclei (FIGS. 1a-c, Table 3). In contrast, NUP98b-EGFP was more diffusely distributed throughout cells, with only 16% of NUP98b-EGFP transfected cells displaying nuclear puncta (FIGS. 1a-c, Table 3). The data demonstrate that the NUP98a FG repeat domain (~39 FG repeats) has potent nuclear localization and condensation capabilities in human cells, which are likely important features that influence the oncogenic potential of NUP98 fusions.

To interrogate the gene regulatory role of NUP98 FG domains at specific loci, the inventors next fused NUP98a or NUP98b FG repeats to the C-terminus of dCas9 (FIG. 1d). When these engineered fusion proteins were targeted to endogenous human promoters, dCas9-NUP98a strongly activated human mRNA expression up to ~100 fold (FIG. 1e). However, dCas9 fused to the shorter NUP98b FG repeat domain only marginally elevated endogenous human promoters, but did activate a minimal viral promoter (miniCMV) better than dCas9-NUP98a (FIG. 7 b) similar to previous studies (Kasper et al., 1999), the significance of which remains to be determined. Further studies revealed that dCas9-NUP98a exhibited a nonlinear relationship between the number of NUP98a FG target sites at a promoter and transcriptional activation (FIG. 1f, FIGS. 7c and 7d). Notably, this activation profile was distinct from other epigenome editing tools (Li et al., 2021; Hilton et al., 2015). For instance, the dCas9-p300 epigenome editor, which is capable of direct histone acetylation, exhibited a linear relationship between the number of gRNA target sites and gene expression (FIGS. 7e and 7f) as the inventors have shown previously (Hilton et al., 2015). This distinct nonlinear gene activation profile displayed by NUP98 FG repeats coupled with its well-established phase separation properties in vitro and in cells (Ahn et al., 2021; Schmidt & Gorlich, 2015), are aligned with emerging models linking phase-separated condensates to transcriptional bursting (Shrinivas et al., 2019; Ma et al., 2021; Hnisz et al., 2017). Therefore, taking these results together with these prevailing models (Shrinivas et al., 2019; Ma et al., 2021; Hnisz et al., 2017), the inventors propose that the NUP98a FG domain directly activates genes in a highly cooperative and phase separation-associated manner.

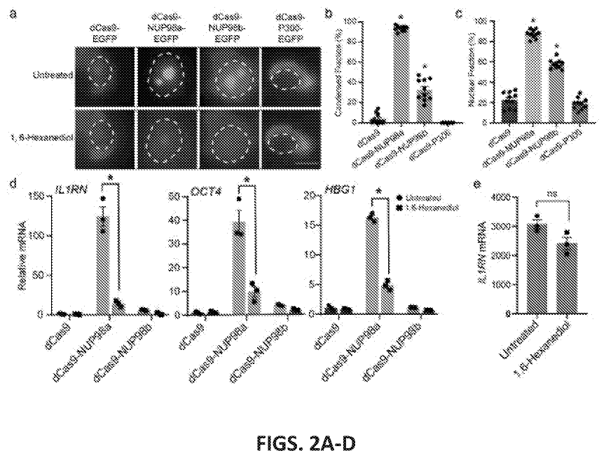

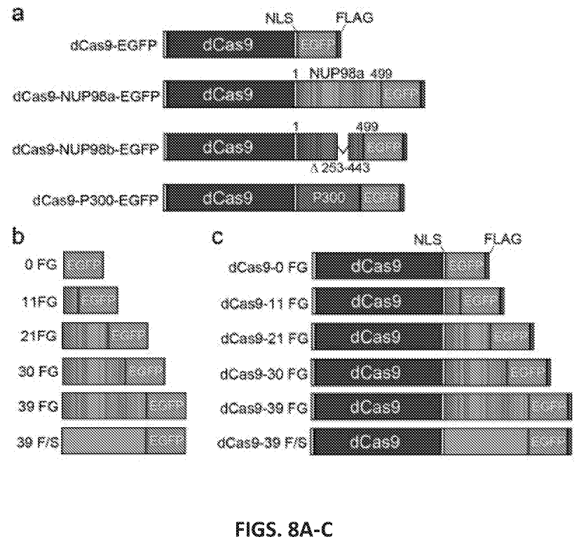

NUP98 FG domains activate human genes in a phase separation-associated manner. To test their hypothesis that the NUP98a FG domain transactivates human promoters in a phase separation-associated manner, the inventors assessed whether dCas9-NUP98a or dCas9-NUP98b fusion proteins formed phase-separated condensates in human cells. To do so, they fused EGFP to the C-terminus of each protein and to dCas9 as a control (FIG. 8a) and observed that dCas9-NUP98a-EGFP and dCas9-NUP98b-EGFP both formed clear nuclear puncta when transiently transfected into HEK293T cells (FIG. 2a; top and FIG. 2b). In contrast, dCas9-p300-EGFP, did not form puncta under the same conditions. Moreover, puncta catalyzed by either dCas9-NUP98a-EGFP or dCas9-NUP98b-EGFP were highly sensitive to 10% 1,6-hexanediol (FIG. 2a; bottom), which is a commonly used reagent to interrogate phase separation behavior (Kroschwald et al., 2017). These live cell imaging data, and the well-established ability of NUP98 to spontaneously phase separate (Ahn et al., 2021; Schmidt & Gorlich, 2015), suggest that these dCas9-NUP98 fusion proteins drive bona fide phase separation in cells.

Interestingly, dCas9-NUP98a-EGFP and dCas9-NUP98b-EGFP also displayed significantly (P<0.05) higher levels of nuclear localization in HEK293T cells compared to dCas9-EGFP alone and dCas9-p300-EGFP. In fact, ~90% of dCas9-NUP98a-EGFP and ~60% of dCas9-NUP98b-EGFP were nuclear-localized, whereas only ~20% of dCas9-EGFP or dCas9-p300-EGFP were present in nuclei (FIG. 2c), suggesting that NUP98 FG repeats are potent facilitators of nuclear localization. To further investigate the link between targeted gene activation and phase separation behavior mediated by dCas9-NUP98a or b, the inventors transiently transfected each into HEK293T cells and added 0.4% of 1,6-hexanediol to cells at 6 hours post-transfection. Even at this relatively low concentration, the ability of dCas9-NUP98a to activate gene expression from human promoters was dramatically diminished (FIG. 2d). However, similar 1,6-hexanediol treatment did not significantly alter the ability of dCas9-p300 to activate gene expression from the IL1RN promoter (FIG. 2e). This high sensitivity of dCas9-NUP98a to a phase separation-disrupting reagent is consistent with its condensation behavior (FIGS. 2a and b) and ability to nonlinearly stimulate gene activation (Shrinivas et al., 2019; Ma et al., 2021; Hnisz et al., 2017) (FIG. 1d, FIGS. 7c-d). Altogether, these collective data establish a link between the ability of NUP98 FG repeats to activate endogenous gene expression and their propensity for phase separation.

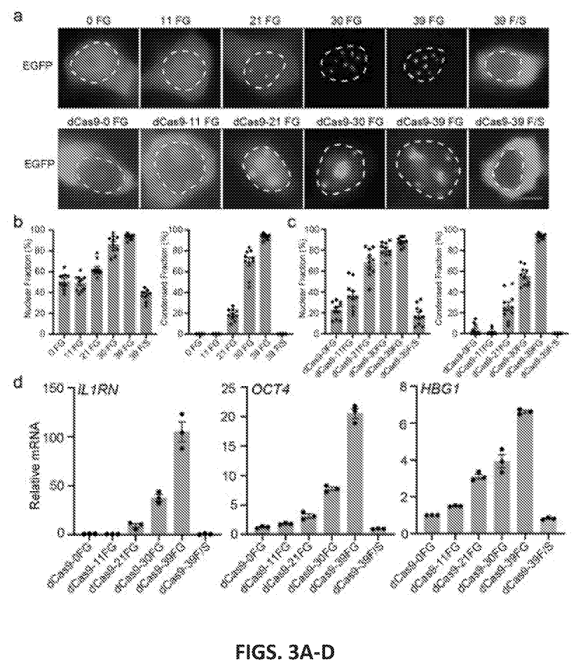

NUP98 FG repeat numbers dictate phase separation and transactivation intensities. Oncogenic NUP98 fusions harbor various numbers of FG repeats (ranging from 19 to 39 FG) in patients (Gough et al., 2011; Hussey et al., 2001; Kasper et al., 1999) which motivated us to test how different FG repeat numbers control NUP98 fusion protein functions. To do so, the inventors first fused a series of FG repeats (spanning 0, 11, 21, 30, and 39 repeats) to EGFP and measured the ability of these fusion proteins to form puncta in cell nuclei (FIG. 2b). Although puncta were observed in HEK293T cell nuclei using 21 FG repeats, a marked increase in nuclear localization and condensation was observed above 30 FG repeats (FIG. 3a; top and FIG. 3b). Similar results were observed when this series of FG repeats and EGFP was fused to dCas9 (FIG. 3a; bottom, FIG. 3c and FIG. 8c). Consistent with this continuum of phase separation behavior, although 21 NUP98 FG repeats activated gene expression when localized to human promoters, potency was enhanced upon localization of 30 or 39 NUP98 FG repeats (FIG. 3d). The inventors also observed that the minimal number of FG repeats (21 FG) required to stimulate phase separation and targeted promoter activation is consistent with the shortest FG repeats (19 FG) detected in NUP98-driven cancers (Kasper et al., 1999). Thus, these data connect the transactivation properties harbored by a critical threshold of NUP98 FG repeats to clinically observed pathologies. Importantly, mutation of phenylalanine to serine (F to S) completely abolished nuclear import, puncta formation, and gene activation capabilities (FIGS. 3A-D, FIGS. 8b and 8c) which further establishes a direct relationship between NUP98 FG repeats, phase separation, and gene activation.

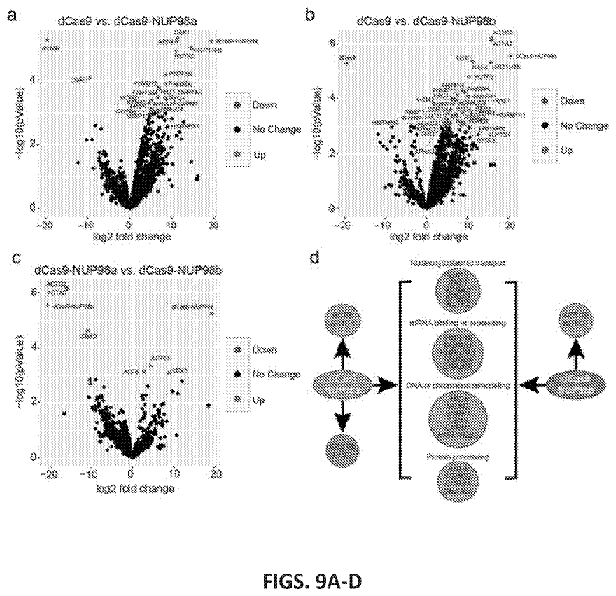

NUP98 FG domains engage scaffolding and gene-regulatory cofactors. To identify the cofactors involved in the condensation, nuclear import, and transactivation properties of dCas9-NUP98a and dCas9-NUP98b, the inventors investigated their protein-protein interactomes using immunoprecipitation of each fusion protein (via the embedded C-terminal FLAG epitopes) followed by Mass Spectrometry (MS). Despite their different FG repeat lengths, transactivation/condensation potencies, and subcellular distributions (FIGS. 1A-F and FIGS. 2A-D), dCas9-NUP98a and b displayed largely similar protein-protein interactomes with only 6 out of 1,450 highly significant (P<0.001) different binding partners identified (FIGS. 9a-c). Compared to dCas9 only control samples, a conserved set of 19 proteins were significantly (P<0.001) enriched in both dCas9-NUP98a and dCas9-NUP98b complexes. These 19 proteins are involved in nucleocytoplasmic transport, mRNA binding and processing, chromatin remodeling, and protein processing (FIG. 9d). Two of these proteins, hnRNPA1 and RAE1, have been shown to interact with diverse NUP98 fusion proteins (Terlecki-Zaniewicz et al., 2021). In particular, hnRNPA1 has a well-documented role in liquid-liquid phase separation (LLPS) (Molliex et al., 2015) and RAE1 is an important factor involved in the NUP98-driven leukemogenesis (Funasaka et al., 2011). Interestingly, dCas9-NUP98a and dCas9-NUP98b were associated with different types of actin. For example, large amounts of ACTG2 and ACTA2 were bound to dCas9-NUP98b but not to dCas9-NUP98a. Conversely, dCas9-NUP98a uniquely bound to CCZ1B/CCZ1, which mediates vacuolar protein trafficking and biogenesis (Delahaye et al., 2014; Nordmann et al., 2010). The similar protein-protein interaction profile between dCas9-NUP98a and dCas9-NUP98b suggests that both short and long NUP98 isoforms lead to the aberrant nuclear events through similar mechanisms, but the unique binding partners likely impact the efficiency of nuclear import, condensation, and subsequent gene regulatory activity within cell nuclei.

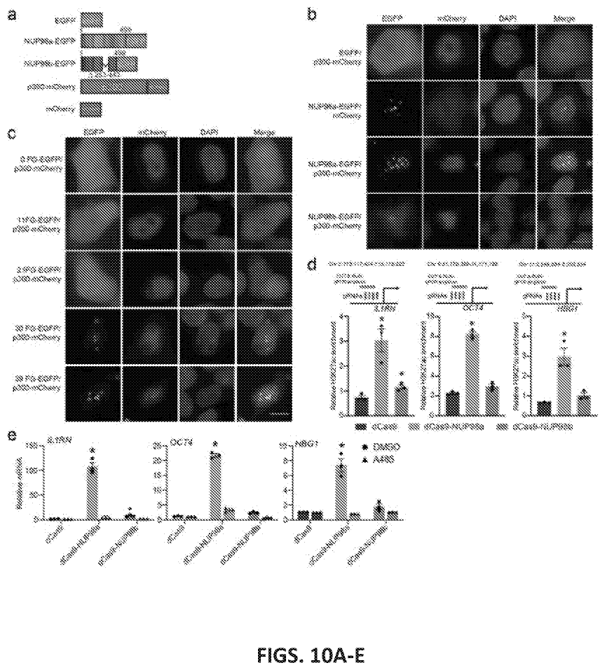

The coactivator p300 has also been shown to interact with the FG domain of NUP98 using immunoprecipitation (Rio-Machin et al., 2017) and Bio-ID (Ahn et al., 2021). However, the MS analysis, and previous MS analyses 4 (Mendes et al., 2020), failed to detect this interaction. To clarify this discrepancy, the inventors investigated the interaction between NUP98 and p300 using fluorescent microscopy as an alternative approach. They overexpressed a full-length p300-mCherry fusion protein along with either NUP98a-EGFP or NUP98b-EGFP fusion proteins in HEK293T cells (FIG. 10a) and observed that full-length p300-mCherry was indeed colocalized with NUP98a and NUP98b-driven condensates (FIG. 10b). They also co-expressed the full length p300-mCherry and various FG repeats in HEK293T cells and observed that p300-mCherry could colocalize with FG-mediated condensates only at FG repeat numbers≥21 repeats (FIG. 10c). Notably, this is also the minimum FG repeat number that showed molecular condensation and gene activation (FIGS. 3A-D). Consistent with this interaction, the inventors observed that targeting dCas9-NUP98a or b to human promoters resulted in increased H3K27ac levels (FIG. 10d) and further, that treatment of cells with the p300 inhibitor A485, abolished the ability of dCas9-NUP98 fusions to activate gene expression from targeted promoters (FIG. 10e). Altogether these data suggest that p300, and associated increases in H3K27ac, are important components of NUP98 FG repeat domain-mediated gene activation and hence oncogenesis.

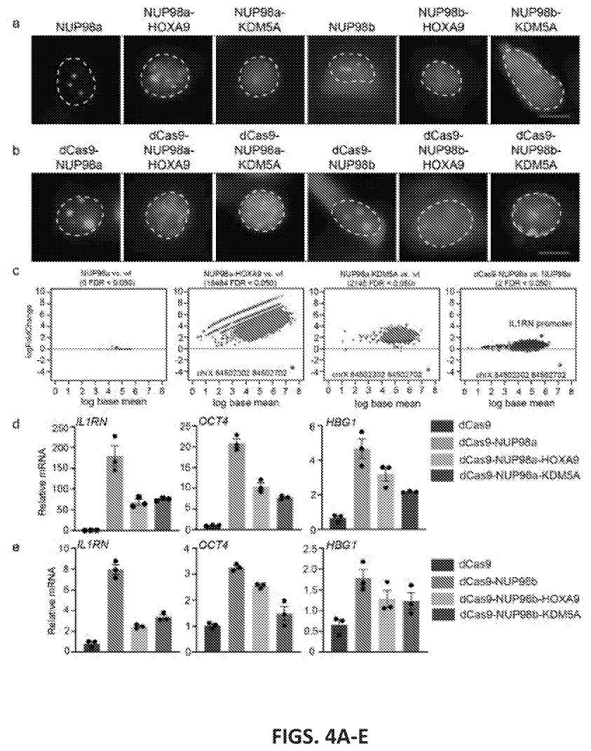

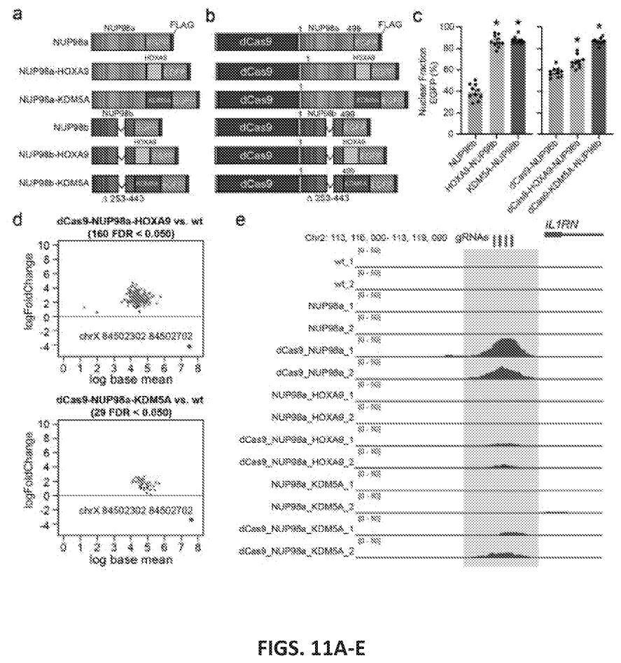

Oncogenic fusion partners control the genomic localization of NUP98 FG repeats. There are two major groups of NUP98 oncogenic fusions in patients based on their C-terminal fusion partner-DNA binding or chromatin binding proteins. HOXA9 (DNA binding) and KDM5A (chromatin binding) are paradigms from these two classes and are frequently observed in leukemia patients with NUP98 fusions (Gough et al., 2011; Bertrums et al., 2023; Chou et al., 2009; Noort et al., 2021). Therefore, to understand how these two major classes of oncogenic NUP98 fusions alter the nuclear localization and/or condensation of NUP98a or b FG repeats, the inventors fused the homeodomain of HOXA9 or PHD domain of KDM5A to the C-terminus of the NUP98a or b FG repeat domains (FIGS. 11a and 11b). Importantly, these fusion architectures match fusion events between NUP98 and HOXA9/KDM5A observed in human cancers (Kasper et al., 1999; Bertrums et al., 2023; Chou et al., 2009; Noort et al., 2021). The fusion of either HOXA9 or KDM5A C-terminal domains (and EGFP) to the N-terminal NUP98a FG repeat domain resulted in a far more diffuse pattern of puncta formation in HEK293T cell nuclei relative to NUP98a fused to EGFP alone (FIG. 4a; left). A similar effect was observed when HOXA9 or KDM5A domains were fused to NUP98b FG repeats (FIG. 4a; right). The inventors also observed that fusion of HOXA9 or KDM5A domains to dCas9-NUP98a or b also altered nuclear localization and puncta morphology (FIG. 4b). Notably, HOXA9 and KDM5A also drastically increased the nuclear localization of NUP98b (FIGS. 4a and b; right, FIG. 11c), suggesting that fusion partner(s) can also stimulate the nuclear import of NUP98b, which the inventors suspect is related to the oncogenic potential of NUP98 fusions, particularly those harboring fewer than ~30 FG repeats.

The inventors next performed ChIP-seq analyses to evaluate whether NUP98a, NUP98a-HOXA9, or NUP98a-KDM5A displayed different profiles of engagement with human genome. Interestingly, NUP98a alone did not display significant levels of interaction with the human genome relative to mock transfected HEK293T cells (FIG. 4c; left panel). However, when domains from HOXA9 or KDM5A were fused to NUP98a, thousands (~18,000 and ~2,000, respectively) of genomic sites became occupied by these respective fusion proteins (FIG. 4c; middle panels). In contrast, when dCas9-NUP98a was targeted to the IL1RN promoter, genomic binding was highly specific compared to NUP98a transfected cells (FIG. 4c; right panel). These results indicate that HOXA9 and KDM5A domains can alter the nuclear localization and condensate morphologies of NUP98 FG repeats in human cell nuclei, and further that dCas9, KDM5A, and HOXA9 domains control the localization of NUP98a within the human genome, ranging from highly specific (dCas9; a single target site) to genome wide (KDM5A and HOXA9; thousands of target sites).

The inventors also tested whether the addition of HOXA9 or KDM5A domains to dCas9-NUP98 fusions would alter the ability to activate endogenous gRNA-targeted promoters. The transcriptional activation capabilities of both dCas9-NUP98a and b were reduced upon fusion of domains from either HOXA9 or KDM5A (FIGS. 4d and 4e), which the inventors attribute to competition between the gRNA-targeted binding of dCas9 and the genomic binding of HOXA9 or KDM5A. In support of this hypothesis, dCas9-NUP98a-HOXA9 and dCas9-NUP98a-KDM5A were bound to many non IL1RN-targeted sites in HEK293T cells (FIG. 11d) and the overall enrichment of dCas9-NUP98a-HOXA9 and dCas9-NUP98a-KDM5A was reduced relative to dCas9-NUP98a at the gRNA-targeted IL1RN site (FIG. 11e).

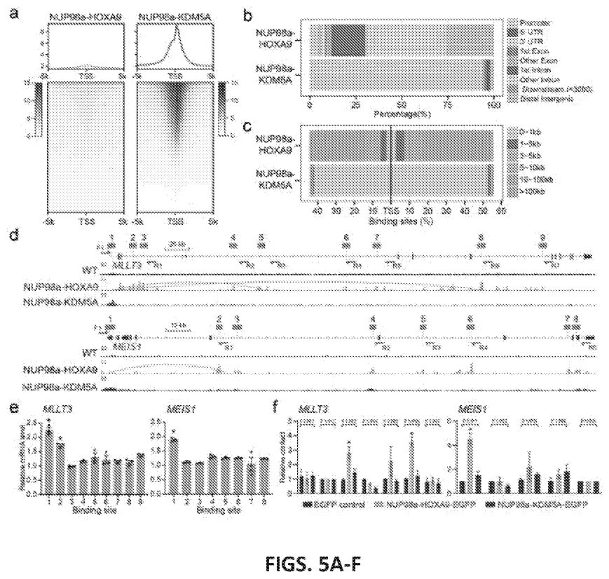

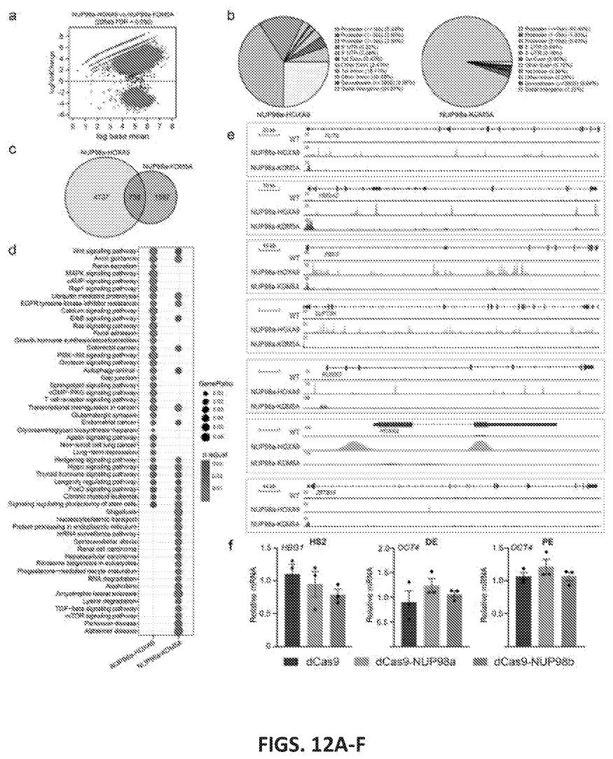

HOXA9 and KDM5A direct NUP98 FG domains to non-overlapping genomic regions. Further analyses of the ChIP-seq data revealed that NUP98a-HOXA9 and NUP98a-KDM5A exhibited non-overlapping binding profiles (FIG. 12a), particularly with respect to human transcription start sites (TSSs; FIG. 5a). ~94% of NUP98a-KDM5A binding sites were located within promoter regions (<1 kb from TSSs) in agreement with previous studies (Zhang et al., 2020). However, only ~5% of NUP98a-HOXA9 bound within 1 kb of annotated TSSs (FIGS. 5a-c, FIG. 12b). Instead, most NUP98a-HOXA9 binding sites (~80%) were located within introns and distal intergenic regions>10 kb from TSSs (only ~2% of NUP98a-KDM5A bound introns or distal intergenic regions). The inventors also found using ChIPseeker (Wang et al., 2022) that NUP98a-HOXA9 and NUP98a-KDM5A bound within annotated promoters, UTRs, exons, introns, downstream or distal intergenic regions of ~5,475 and ~2,325 genes, respectively, however only ~738 of these genes were shared between fusions (FIG. 12c). Kyoto Encyclopedia of Genes and Genomes (KEGG) (Kanehisa et al., 2023) analyses also revealed that the binding of NUP98a-HOXA9 and NUP98a-KDM5A was associated with largely non-overlapping biological pathways. However, one important exception was a shared association with genes involved in oncogenic pathways including chronic myeloid leukemia, Wnt signaling, colorectal cancer, transcriptional misregulation in cancer, and endometrial cancer (FIG. 12d).

Within the shared transcriptional misregulation in cancer KEGG pathway, several known leukemia genes (MLLT3, MEIS1, FUT8, HMGA2, PBX3, SUPT3H, RUNX2 and HOXA9, ZBTB16) (Liu et al., 2018) were bound by NUP98a-HOXA9 and NUP98a-KDM5A, albeit with different binding profiles between the proteins. For instance, NUP98a-HOXA9 was highly enriched across multiple binding sites spanning entire gene bodies at these leukemia genes, whereas NUP98a-KDM5A showed highly localized near their promoters (FIG. 5d, FIG. 12e). Interestingly, the NUP98a-KDM5A binding pattern at promoters closely mimicked the pattern of engineered binding using dCas9-NUP98 fusions at human promoters (FIGS. 1A-3D). In fact, the inventors were able to synthetically mimic NUP98a-KDM5A binding at MLLT3 and MEIS1 by targeting dCas9-NUP98a to their promoters, which consequently activated the expression of these genes (FIG. 5e). Therefore, these data indicate that localizing high levels of NUP98 FG repeats to human promoters is one method that drives pathological gene expression in NUP98-associated cancers.

In contrast to NUP98a-KDM5a, the inventors were unable to stimulate MLLT3 or MEIS1 by targeting dCas9-NUP98 to distal genomic segments matching the sites occupied by NUP98a-HOXA9, nor by targeting known intergenic OCT4 or HBG1 enhancers (Hilton et al., 2015; Deng et al., 2014; Carter et al., 2002) (FIG. 5e, FIG. 12f). Considering that large numbers of NUP98a-HOXA9 often decorate long stretches (>5 kb) of genomic DNA to produce a “super-enhancer” like phenomenon in leukemias (Ahn et al., 2021) (FIG. 5d and FIG. 12e), the inventors propose that the binding site numbers and densities, as well as genomic locations, all underlie the leukemogenic potential of NUP98a-HOXA9. NUP98a-HOXA9 in particular can modulate chromosomal looping though large numbers of binding sites across long genomic regions (Ahn et al., 2021). Therefore, the inventors hypothesize that NUP98-HOXA9 may control transcription through the creation of chromatin contacts between promoters and other bound sites in introns. To test this hypothesis, they performed 3C-qPCR in HEK293T cells transduced with EGFP, NUP98-HOXA9, or NUP98-KDM5A. These experiments demonstrated that NUP98a-HOXA9 catalyzed chromatin contacts between NUP98a-HOXA9 sites near the MLLT3 and MEIS1 promoters and other NUP98-HOXA9 bound sites at introns (FIGS. 5d and 5f). Changes in chromosomal contacts were not observed in NUP98a-KDM5A transfected cells. These data suggest that the genomic reorganization mediated by high densities of NUP98 FG binding across large genomic regions is another method to stimulate oncogenic gene expression in NUP98-associated cancers.

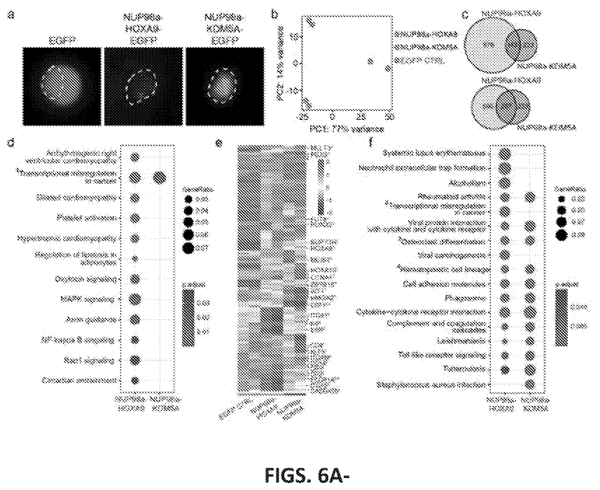

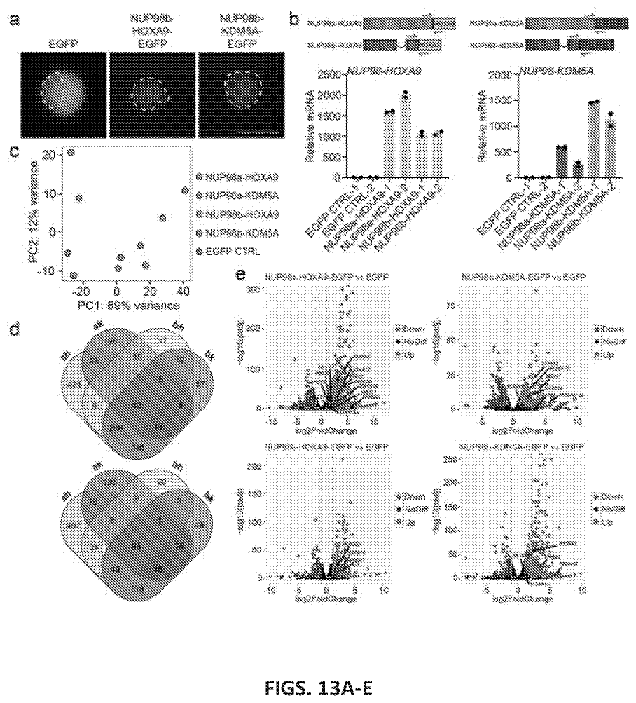

Different NUP98 fusions converge upon the activation of a core leukemogenic gene expression program in primary human HSCs. To investigate the effects of NUP98 fusions in a more clinically proximal context, the inventors delivered NUP98a-HOXA9-EGFP, NUP98a-KDM5A-EGFP, NUP98b-HOXA9-EGFP, or NUP98b-KDM5A-EGFP into primary mobilized human peripheral blood CD34+ hematopoietic stem cells (HSCs) using lentiviral transduction. Consistent with the inventors' observations in HEK293T cells, all NUP98-HOXA9/KDM5A fusion proteins localized to the nuclei of HSCs and NUP98a-HOXA9-EGFP and NUP98a-KDM5A-EGFP formed clear nuclear puncta (FIG. 6a and FIG. 13a). They sorted EGFP positive HSCs and validated the expression of NUP98 fusions using RT-qPCR (FIG. 13b). Next, they performed RNA-seq on these transduced HSCs and found that all NUP98 fusions displayed distinct transcriptomes relative to one another and to EGFP control transduced cells (FIG. 6b, FIG. 13c). In fact, only ~17% (410 out of 2,434 total DEGs) of all differentially expressed genes (DEGs) overlapped between HSCs transduced with NUP98a-HOXA9 and NUP98a-KDM5A, and only ~6% (148 out of 2,590 total DEGs) overlapped between all HSCs transduced with NUP98 fusions (FIG. 6c, FIGS. 13d and 13e).

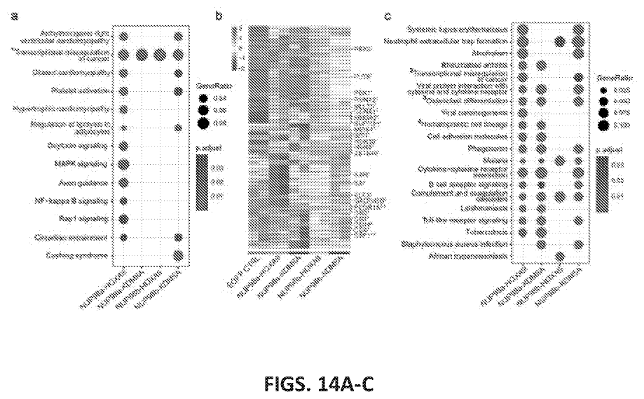

Further comparative RNA-seq analyses revealed that all tested NUP98 fusions led to substantial perturbations in the transcriptional misregulation in cancer KEGG pathway regardless of NUP98 isoform or fusion partner (FIGS. 6d and 6e, FIGS. 14a and 14b). In particular, NUP98a-HOXA9 and NUP98a-KDM5A markedly upregulated the expression of the HOXA9/MEIS1 transcriptional program (Collins & Hess, 2016) as well as several other leukemia-associated transcription factors (e.g., RUNX2 (Matthijssens et al., 2021), WT1 (Rampal & Figueroa, 2016), ZBTB16 (Suliman et al., 2012), and MLLT3 (Calvanese et al., 2019)) that together are responsible for the proliferation and aberrant transcription of leukemia cells. Further, a key cytokine in the HSC lineage commitment, CSF1 (Stanley, 2009), was downregulated in HSCs transduced with either NUP98a-HOXA9 and NUP98a-KDM5A (FIGS. 6e and 6f, FIGS. 8b and 8c), consistent with evidence that NUP98 fusions can disrupt human HSC differentiation programs (Takeda et al., 2006). Notably, the inventors observed important exceptions to this shared core leukemogenic transcriptional program between NUP98a-HOXA9 and KDM5A, in that only NUP98a-HOXA9 triggered the expression of additional oncogenes (e.g PBX3 (Li et al., 2013), SUPT3H (Nakamura et al., 2015) and FUT8 (Agrawal et al., 2017)) and decreased the expression of several tumor suppressor genes (e.g., IL6 (Li et al., 2015), GADD45B (Liebermann, 2022) and KLF3 (Sachdeva et al., 2015); FIG. 6e), which likely act synergistically to facilitate leukemic transformation in NUP98-HOXA9-associated cancers.

Given the ChIP-seq binding profiles of NUP98a-HOXA9/KDM5A in HEK293T cells (FIG. 5d and FIG. 12e), these oncogenic fusions likely directly induce the aberrant transcription of HOXA9, MEIS1, RUNX2, ZBTB16, and MLLT3 in human HSCs, which likely indirectly causes the downregulation of hematopoietic cytokines and tumor suppressors (e.g., CSF1, IL6, GADD45B and KLF3). Therefore, despite their discrete binding profiles and largely non-overlapping transcriptomic dysregulation, NUP98-HOXA9 and NUP98-KDM5A fusions converge upon a core leukemogenic program that appears to be driven by inappropriate direct activation and disruption of HSC proliferation and differentiation programs.

The inventors' previous work demonstrated that NUP98 FG domains strongly induce dCas9 fusion protein condensate at specific genomic loci when fused to CRISPR/dCas9. To take advantage of this property, they hypothesized that fusion of NUP98 FG domains to current CRISPR/Cas9 based epigenome or genome editing tools may recruit multiple copy of the epigenome or genome editing molecules, and thus enhance their efficiencies. However, the dCas9 fusion proteins containing the endogenous NUP98 FG repeats (dCas9-NUP98a or dCas9-NUP98b) strongly bind to the RNA export factor 1 (RAE1), leading to mislocalization and haploinsufficiency of RAE1 that has been linked with aneuploidy.

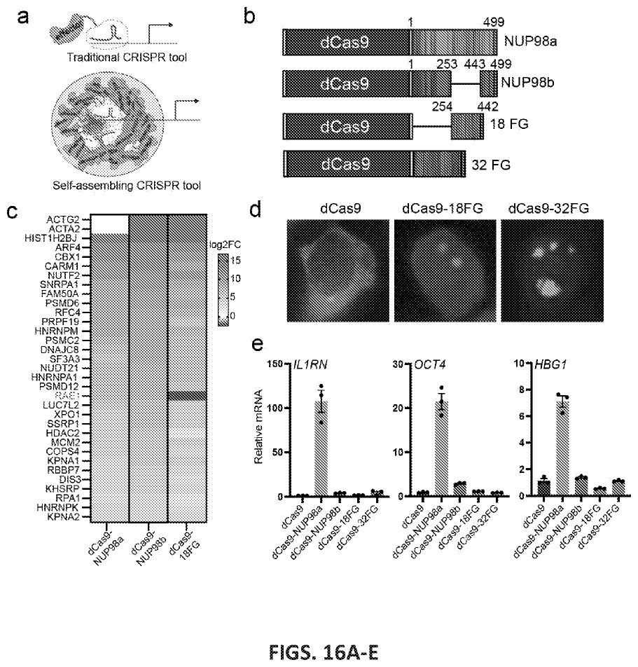

To develop more efficient but safer CRISPR/Cas9 based epigenome or genome editing condensates, the inventors engineered a new NUP98 FG fusion (dCas9-18FG) that lose interaction with RAE1 (FIGS. 16A and 16C). Interestingly, dCas9-18 FG still tends to self-assembling but is insufficient to activate human genes when targeting at the human promoters (FIGS. 16B and 16D). To increase the self-assembling ability, they also engineered another synthetic FG domain (32 FG) that contains a sequence similar to 18 FG. Those synthetic FG domains are better candidates to enhance the efficiency of the current CRISPR tools. As such, the inventors were able to engineer a synthetic but safer FG domain that does not bind to RAE1.

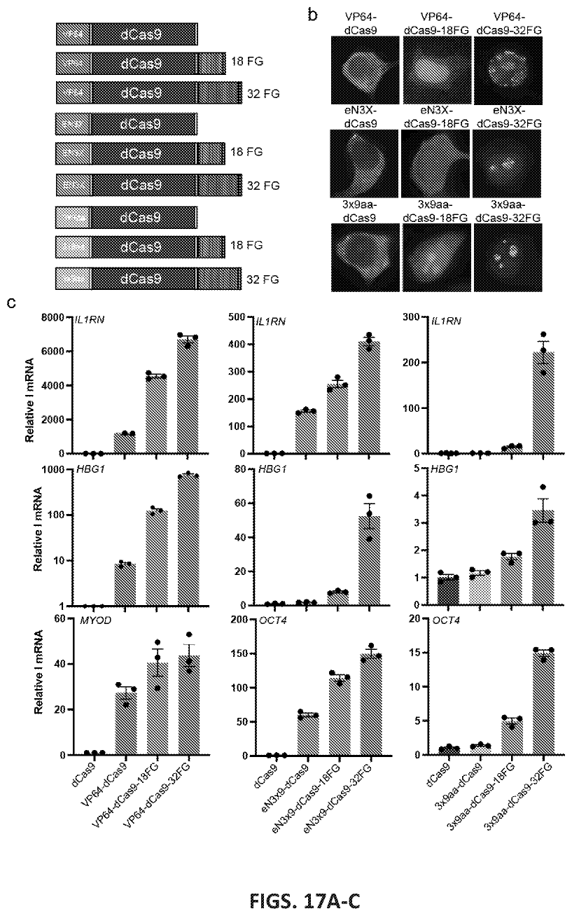

The inventors also fused the synthetic FG domain (18FG and 32FG) to three of the current CRISPR activation (CRISPRa) tools (VP64-dCas9, eN3x-dCas9 and 3x9aa-dCas9) (FIG. 17A). They noticed that fusion of FG induces the condensation of the new fusion proteins (VP64-dCas9-18FG-EGFP, VP64-dCas9-32FG-EGFP, eN3x-dCas9-18FG-EGFP, eN3x-dCas9-32FG-EGFP, 3x9aa-dCas9-18FG-EGFP and 3x9aa-dCas9-32FG-EGFP) in nuclei (FIG. 17B). As they expected, compared with the traditional CRISPRa tools (VP64-dCas9, eN3x-dCas9 and 3x9aa-dCas9), the transcriptional activation capacities of the new FG containing fusions were hugely increased (FIG. 17B). Thus, synthetic FG domains amply the transactivation efficiency of CRISPRa tools.

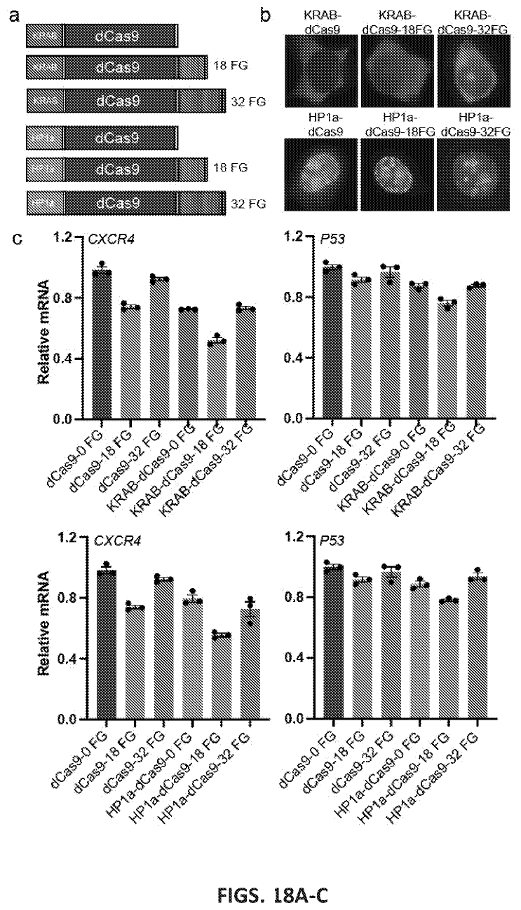

The inventors next fused the synthetic FG domain (18FG and 32FG) to two of the current CRISPR repressor tools (KRAB-dCas9 and HP1a-dCas9), and noticed fusion of FG doesn't help condensation of the new fusion proteins. However, compared with the traditional CRISPRi tools (KRAB-dCas9 and HP1a-dCas9), the transcriptional repression was intensified by KRAB-dCas9-18 FG and HP1a-dCas9-18FG. See FIGS. 18A-C. Thus, synthetic FG domains intensify the transcriptional repression of current CRISPRi tools.

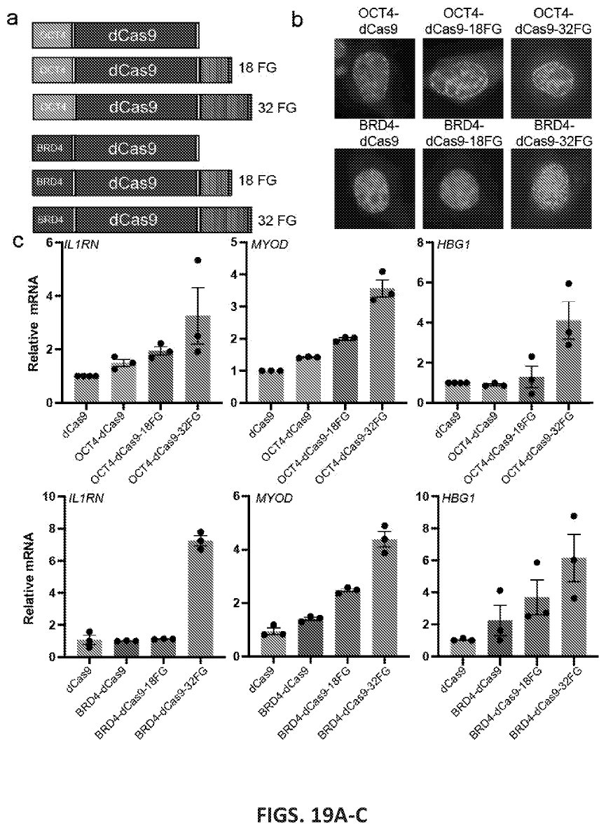

The inventors fused the synthetic FG domain (18FG and 32FG) to two of synthetic transcriptional factors (OCT4-dCas9 and BRD4-dCas9) that spontaneously form transcriptional condensates. The inventors noticed fusion of 18 or 32 FG domains to OCT4-dCas9 or BRD4-dCas9 condensate as well as OCT4-dCas9 and BRD4-dCas9. However, the FG domain containing fusions activate human gene much better when targeting at the promoter regions. See FIGS. 19A-C. Thus, synthetic FG domains engineered the transcriptional condensates and increase the transactivation of CRISPR/Cas9 based transcriptional condensates.

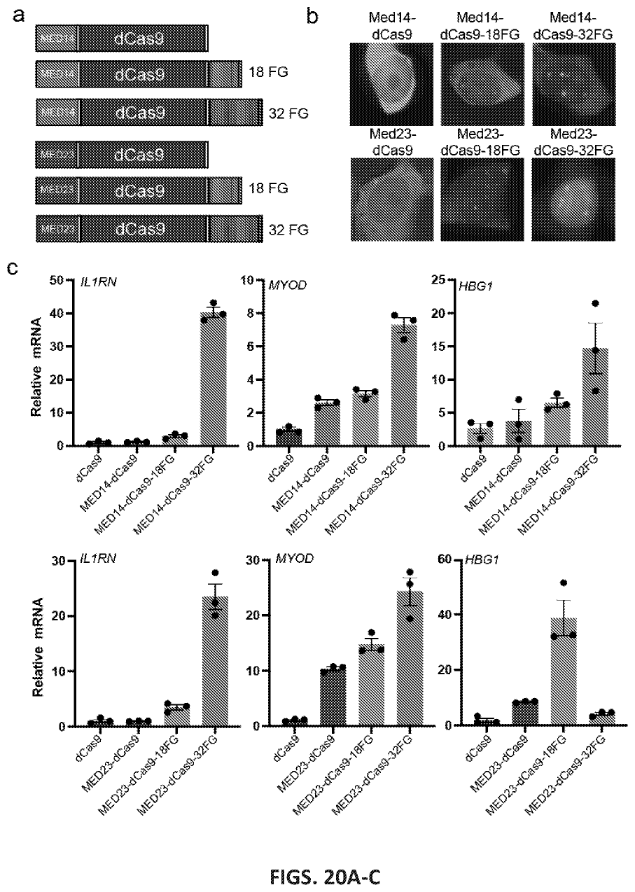

The inventors fused the synthetic FG domain (18FG and 32FG) to two of the synthetic cofactors (MED14-dCas9 and MED23-dCas9) that don't condensate in HEK193T cells. They noticed fusion of 18 or 32 FG domains to MED14-dCas9 or MED23-dCas9 induces the condensation of new fusions. Furthermore, the FG domain containing fusions start to activate human genes when targeting at the promoter regions. See FIGS. 20A-C. Thus, synthetic FG domains promote the condensation of transcriptional co-factors and induce human gene activation.

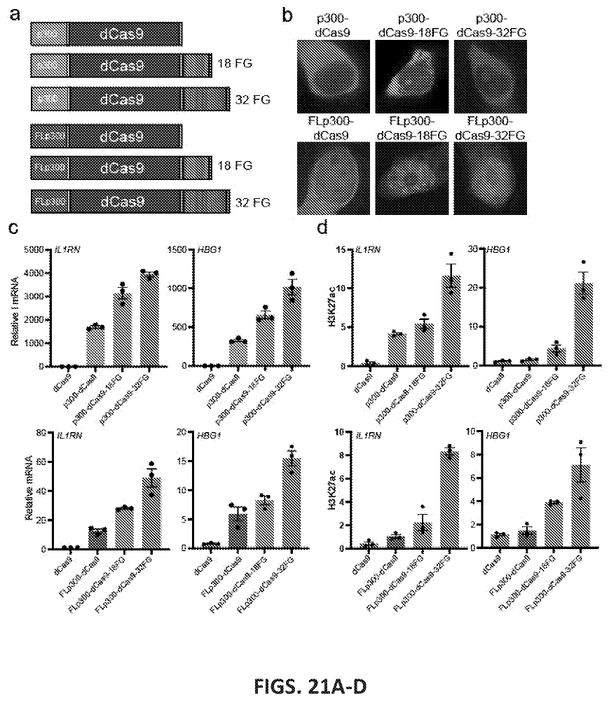

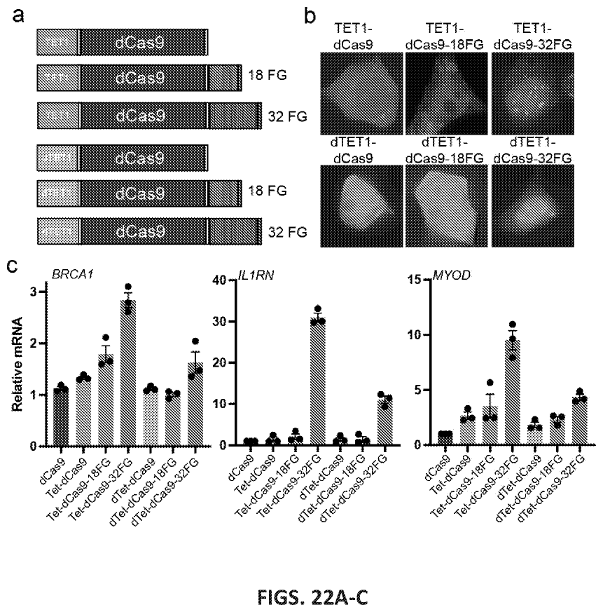

The inventors also fused the synthetic FG domain (18FG and 32FG) to histone acetylation tool (p300-dCas9 and full-length p300-dCas9). They noticed fusion of FG domains (18 FG and 32 FG) promote the condensation of full-length p300-dCas9. As they expected, compared with the p300-dCas9 or full-length p300-dCas9, the transcriptional activation capacities of new fusions containing the FG domain were increased and the K27ac enrichment at the targeting promoter also increased. See FIGS. 21A-D. Thus, synthetic FG domains increase the histone modification efficiency.

The inventors fused the synthetic FG domain (18FG and 32FG) to DNA demethylation tool (TET1-dCas9) and its deactivated version (dTET1-dCas9). They noticed fusion of FG domains (18 FG and 32 FG) promote the condensate of the new fusion proteins. As they expected, compared with the TET1-dCas9 or dTET1-dCas9, the transcriptional activation capacities of new fusions containing the FG domain were increased. Thus, synthetic FG domains induce an increase in the DNA demethylation efficiency of TET1-dCas9.

TABLE 3
Percentage of cells containing punctas in the EGFP,
NUP98a-EGFP, NUP98b-EGFP transfected HEK293T

| | EGFP | NUP98a-EGFP | NUP98b-EGFP |
|---|---|---|---|
| Total cells | 100 | 100 | 100 |
| Cells with puncta | 0 | 98 | 16 |
| Percentage (%) | 0% | 98% | 16% |

### Example 3—Discussion

Many diseases are driven by aberrant biomolecular condensation catalyzed by oncogenic fusion proteins (Niu et al., 2023), which underscores the need for new mechanistic insights into these phenomena. Therefore, understanding the oncogenic effects of NUP98 FG fusion is not only critical for the development of new treatments for NUP98-driven cancers, but informative for understanding the pathology of other oncogenic fusions containing intrinsically disordered regions (IDRs). Here, the inventors dissect the transcriptional regulatory role of NUP98 FG repeats at specific sites and at the genome-wide scale and demonstrate that aberrant biomolecular condensation and genomic engagement of NUP98 FG repeat bearing fusions leads to a dysregulated transcriptome converging upon hallmarks of leukemic transformation.

Once phase separation-competent proteins exceed a critical concentration, they can spontaneously phase separate (Alberti et al., 2019). The FG domains from several nuclear pore proteins have very low measured critical concentrations in vitro (1-35 μM) (Delahaye et al., 2014). In this study, increasing the gRNA binding sites for dCas9-NUP98a, likely increases the local concentration of NUP98a FG repeats at targeted loci, which in turn likely exceeds the critical concentration required for NUP98 FG repeat-driven phase separation. The inventors found dCas9-NUP98a displayed a nonlinear gene regulatory curve at targeted promoters, which is a defining feature of transcriptional condensates (Shrinivas et al., 2019; Ma et al., 2021; Hnisz et al., 2017). Mechanistically, they observed that NUP98 FG repeats co-condensate with other IDR-enriched coactivators (e.g., p300), and result in increased H3K27ac enrichment at targeted promoters. These results further demonstrate that the NUP98 FG repeats utilize multivalent interactions with IDRs from other transcriptional cofactors to drive phase separated transcriptional condensates and simulate gene expression.

Thermodynamically, there is also a log-linear relationship between NUP98 FG repeat numbers and partition coefficients, wherein higher FG repeat numbers correspond to higher partition coefficients and lower Gibbs free energies (Ng & Gorlich, 2022). In agreement with this thermodynamic model, the inventors found that FG repeat numbers are positively correlated with the levels of both phase separation behavior and gene activation from human promoters. However, the inventors note that this correlation may not apply to all promoters. For example, the data and previous work indicate that shorter FG domains (i.e., NUP98b; 21 repeats) can stimulate transcription better than longer FG repeat domains (i.e., NUP98a; 39 repeats) at synthetic, virally derived promoters (Kasper et al., 1999). Regardless, it is likely that both long and short NUP98 FG repeats contribute to oncogenic effects through transcriptional activation when aberrantly fused to chromatin or DNA binding domains. For example, although most oncogenic NUP98 fusions in patients contain more than 30 FG repeats (Gough et al., 2011), oncogenic fusion events can arise via fewer repeats (e.g., NUP98-LEDGF; 23 FG repeats (Hussey et al., 2001) or NUP98-HOXA9; 19 FG repeats (Kasper et al., 1999)). The inventors' transcriptomic analyses in primary human HSCs demonstrate that the shorter NUP98b fusions can stimulate oncogenic gene expression patterns, albeit to a lesser extent than the longer NUP98a harboring fusions. Interestingly, the inventors observed that fusion partners (HOXA9 and KDM5A) can assist with the transport of shorter FG repeats (NUP98b; 21 FG repeats) into the nucleus, which may enhance subsequent oncogenic processes in cancers where fewer NUP98 FG repeats are observed.

IDR-driven phase separation behavior has also been linked to transcriptional control at super enhancers in mammalian cells (Hnisz et al., 2017). Together, the well-defined phase separation property of NUP98 FG repeats (Schmidt & Gorlich, 2015), the super enhancer-like binding pattern, and chromatin looping catalyzed by NUP98a-HOXA9 identified here and previously (Ahn et al., 2021), all suggest that NUP98a-HOXA9 mimics this super enhancer-like transcriptional control during leukemic transformation. The inventors' studies demonstrate that the super enhancer like transcriptional control of NUP98a-HOXA9 fusions is likely very intense due to the high-density of FG repeats bound across long stretches (>5 kb) of genomic DNA. For instance, NUP98a-HOXA9 strongly activates the PBX3 (Li et al., 2013), SUPT3H (Nakamura et al., 2015) and FUT8 (Agrawal et al., 2017) proto-oncogenes through dense clusters of binding across these respective gene bodies, whereas NUP98a-KDM5A is unable to activate these genes through localized promoter binding. This strict dependence on the binding site numbers and densities of transcriptional factors over a long genomic window is a hallmark of super enhancers (average window ~8.7 kb (Whyte et al., 2013)), and may explain why targeting dCas9-NUP98a to short (~500-1,000 bp) “typical” enhancers or to short segments of NUP98a-HOXA9 bound introns was insufficient to stimulate transcription. In addition, experiments using dCas9-NUP98a demonstrate that promoters appear to be more sensitive and permissive to the transcriptional effects of NUP98 FG repeats. Moreover, the data suggest that NUP98-KDM5A may function similarly to dCas9-NUP98a, in which high levels of focused enrichment at target promoters leads to phase separation and increased gene expression.

HOXA9 and KDM5A are representatives of the two major classes of NUP98 fusion partners: homeodomain containing (HOXA9) and chromatin-modifying (KDM5A) proteins (Gough et al., 2011). Therefore, the genomic binding profiles and transcriptional regulatory mechanisms of NUP98-HOXA9 and NUP98a-KDM5A likely exemplify the oncogenic processes of the myriad of other NUP98 fusion proteins observed in cancers. For instance, the homeodomain is found in numerous other NUP98 fusion contexts (e.g., HOXA11/13, HOXC11/13, HOXD11/13, PMX1/2 and HHEX) (Gough et al., 2011). Furthermore, NSD1, a chromatin-modifying protein is one of the most frequent oncogenic NUP98 fusion partners and also show substantial binding around promoter regions (Ren et al., 2022), suggesting that NUP98-NSD1 fusions act similarly to NUP98-KDM5A and trigger leukemic transformation through aberrant focal condensation at promoters.

These comparative studies identify a set of shared leukemia gene expression programs between two major groups of NUP98 fusions. Therefore, it is tempting to speculate that NUP98-based malignancies may have common therapeutic dependencies that could enable potent and ubiquitous new treatment options. The data and previous studies (Bertrums et al., 2023; Collins & Hess, 2016) establish that HOXA9-based regulatory components, such as HOXA9 and MEIS1 are commonly triggered by NUP98-HOXA9, NUP98-KDM5A and many other leukemia fusions. Nevertheless, the inventors note that NUP98-HOXA9 and NUP98-KDM5A have many non-overlapping effects, and thus unique therapeutic strategies could also be tailored for specific NUP98 fusions (Bertrums et al., 2023). Altogether, this study demonstrates that the FG repeats from NUP98a can drive biomolecular condensation and activate human genes, which in turn clarifies the biological role of NUP98 FG fusions in oncogenesis. Further interrogation of these processes could be useful for the treatment of NUP98-driven human cancers, as well as other cancers caused by inappropriate fusions between IDRs and proteins that bind the human genome (Niu et al., 2023). Moreover, the important biomolecular properties of the NUP98 FG repeats and other IDRs could be harnessed to enable new bioengineering capabilities (Liu et al., 2023; Ma et al., 2023; Kim et al., 2023).

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### V. REFERENCES

The following references, to the extent that they provide exemplary procedural or other details supplementary to those set forth herein, are specifically incorporated herein by reference.
Agrawal et al., Cancer Cell 31, 804-819 e807 (2017).
Ahn et al., Nature 595, 591-595, (2021).
Alberti et al., Cell 176, 419-434 (2019).
Arai et al., Blood 89, 3936-3944 (1997).
Bertrums et al., Haematologica 108, 2044-2058 (2023).
Calvanese et al., Nature 576, 281-286 (2019).
Carter et al., Nat Genet 32, 623-626 (2002).
Chandra et al., Cancer Discov 12, 1152-1169 (2022).
Chou et al., Leukemia 23, (2009).
Collins & Hess, Curr Opin Hematol 23, 354-361 (2016).
Delahaye et al., Mol Biol Cell 25, 1073-1096 (2014).
Deng et al., Cell 158, 849-860 (2014).
Escobar et al., ACS Synth Biol 11, 3239-3250 (2022).
Franks & Hetzer, Trends Cell Biol 23, 112-117, (2013).
Funasaka et al., Cell Cycle 10, 1456-1467 (2011).
Gough et al., Blood 118, 6247-6257 (2011).
Hagege et al., Nat Protoc 2, 1722-1733 (2007).
Hilton et al., Nat Biotechnol 33, 510-517 (2015).
Hnisz et al., Cell 169, 13-23 (2017).
Hussey et al., BMC Genet 2, 20 (2001).
Kanehisa et al., Nucleic Acids Res 51, D587-D592 (2023).
Kasper et al., Mol Cell Biol 19, 764-776 (1999).
Kim et al., Nat Biotechnol 37, 907-915 (2019).
Kim et al., Sci Adv 9, eadg1123 (2023).
Kroschwald et al., Matters (2017).
Langmead & Salzberg, Nat Methods 9, 357-359 (2012).
Li et al., Blood 121, 1422-1431 (2013).
Li et al., Blood 126, 766-778 (2015).
Li et al., Nat Commun 12, 896 (2021).
Liao et al., Bioinformatics 30, 923-930 (2014).
Liu et al., Database (Oxford) 2018 (2018).
Liu et al. Protein Cell (2023).
Liebermann, Adv Exp Med Biol 1360, 41-54 (2022).
Love et al., Genome Biol 15, 550, (2014).
Ma et al., Mol Cell 81, 1682-1697 e1687 (2021).
Ma et al., Nucleic Acids Res 51, 5271-5284 (2023).
Matthijssens et al., J Clin Invest 131 (2021).
Mendes et al., Cells 9, (2020).
Michmerhuizen et al., Blood 136, 2275-2289 (2020).
Molliex et al., Cell 163, 123-133 (2015).
Nakamura et al., Blood Cancer J 5, e301 (2015).
Ng & Gorlich, Nat Commun 13, 6172 (2022).
Niu et al., MedComm (2020) 4, e223 (2023).
Nordmann et al., Curr Biol 20, 1654-1659 (2010).
Noort et al., Haematologica 106, 630-634 (2021).
Rampal & Figueroa, Haematologica 101, 672-679 (2016).
Ramirez et al., Nucleic Acids Res 44, W160-165 (2016).
Ren, Z. et al. Proc Natl Acad Sci USA 119 (2022).
Ritchie et al., Nucleic Acids Res 43, e47 (2015).
Rio-Machin et al., Leukemia 31, 2000-2005 (2017).
Robinson et al., Bioinformatics 39, (2023).
Ross-Innes et al., Nature 481, 389-393 (2012).
Sachdeva et al., Cancer Lett 369, 202-211 (2015).
Schmidt & Gorlich, Elife 4, (2015).
Shrinivas et al., Mol Cell 75, 549-561 e547 (2019).
Stanley, Cell Stem Cell 5, 234-236 (2009).
Suliman et al., Front Oncol 2, 74 (2012).
Takeda et al., Cancer Res 66, 6628-6637 (2006).
Terlecki-Zaniewicz et al., Nat Struct Mol Biol 28, 190-201 (2021).
Wang et al., Curr Protoc 2, e585 (2022).
Whyte et al., Cell 153, 307-319 (2013).
Wu et al., Innovation (Camb) 2, 100141 (2021).
Zhang et al., Genome Biol 9, R137 (2008).
Zhang et al. Nat Commun 11, 3339 (2020).

1. A fusion protein comprising (a) a phenylalanine/glycine (FG) domain comprising repeating patterns of phenylalanine (F) and glycine (G) and (b) a Cas9 protein.

2. The fusion protein of claim 1, wherein the FG domain does not bind to RNA export factor 1.

3. The fusion protein of claim 1 or claim 2, further comprising an effector protein, such as a transcription activator, a transcription co-factor, a transcription repressor, a histone acetylase, or a DNA demethylase.

4. (canceled)

5. The fusion protein of claim 3, wherein the effector protein is p300, KRAB, VP64, TET1 (e.g., dTET1), eN3x, HP1a-d, BRD4, OCT4, MED14, or MED23.

6. The fusion protein of claim 1, wherein the Cas9 protein is a CRISPR-based transcriptional activator, a CRISPR-based transcriptional repressor, or a CRISPR-based epigenome editor, a CRIPSR-based nucleases, a CRIPSR-base editor, a CRIPSR-based prime editor, a CRIPSR-based transposase, a dCas9-modifier of DNA methylation, a dCa9-modifier of histone methylation, or a dCas9-modifier of histone phosphorylation.

7. The fusion protein of claim 1, wherein the FG domain comprises 10 to 50 F/G repeats, 18 F/G repeats, 21 F/G repeats, 32 F/G repeats, or 39 F/G repeats.

8. The fusion protein of claim 1, wherein the FG domain comprises or consists of an amino acid sequence at least 95%, at least 96%, at least 97%, at least 98%, at least 99%, or 100% identical to the amino acid sequence of SEQ ID NO: 144 or 147.

9. A pharmaceutical composition comprising the fusion protein of claim 1.

10. A nucleic acid encoding the fusion protein of claim 1.

11. A cell comprising the fusion protein of claim 1.

12. A method of transporting a Cas9 protein into a cell nucleus comprising introducing the fusion protein of claim 1 into a cell.

13. A method of modifying the phenotype of a cell comprising introducing the fusion protein of claim 1 into the cell, along with a gRNA.

14. The method of claim 13, wherein the introduction of the fusion protein and gRNA into the cell causes targeted modification of the epigenome of the cell.

15. The method of claim 13, wherein the introduction of the fusion protein and gRNA into the cell causes transcription activation or repression of a target gene.

16. The method of claim 13, wherein the introduction of the fusion protein and gRNA into the cell causes targeted histone acetylation or DNA demethylation.

17. A kit comprising the fusion protein of claim 1, optionally further comprising a gRNA.

18. A method of transporting a Cas9 protein into a cell nucleus comprising introducing the nucleic acid of claim 10, into a cell.

19. A method of modifying the phenotype of a cell comprising introducing the nucleic acid of claim 10, into the cell, along with a gRNA.

20. A cell comprising the nucleic acid of claim 10.

21. A kit comprising the nucleic acid of claim 10, optionally further comprising a gRNA.
